(19) United States
(12) Patent Application Publication
(10) Pub. No.: US 2023/0210597 A1
(43) Pub. Date: Jul. 6, 2023

(54) IDENTIFICATION OF BONE AREAS TO BE REMOVED DURING SURGERY

(71) Applicant: Howmedica Osteonics Corp., Mahwah, NJ (US)

(72) Inventors: Vincent Abel Maurice Simoes, Locmaria Plouzané (FR); Florence Delphine Muriel Maillé, Locmaria Plouzané (FR); Jean Chaoui, Lyon (FR)

(21) Appl. No.: 17/928,191

(22) PCT Filed: Apr. 29, 2021
(86) PCT No.: PCT/US2021/029904

§ 371 Date: Nov. 28, 2022

Related U.S. Application Data
(60) Provisional application No. 63/034,092, filed on Jun. 3, 2020.

(51) Int. Cl. A61B 34/10; G06T 19/00

(52) U.S. Cl. CPC A61B 34/10; G06T 19/006; A61B 2034/105; G06T 2210/41

(57) ABSTRACT

Techniques are described for identifying bone areas for removal during a medical procedure. Processing circuitry may generate a virtual model of an anatomical object with the bone areas removed and one or more indications for the bone areas that are to be removed. During surgery, the processing circuitry may receive image data and update the one or more indications as the bone areas are removed. The processing circuitry may generate the virtual model and the bone areas for visual overlay on the actual anatomical object.

This application claims the benefit of U.S. Provisional Pat. Application No. 63/034,092, filed 3 Jun. 2020, the entire contents of which are incorporated herein by reference.

### BACKGROUND

Surgical joint repair procedures involve repair and/or replacement of a damaged or diseased joint. A surgical joint repair procedure, such as joint arthroplasty as an example, may involve replacing the damaged joint with a prosthesis that is implanted into the patient’s bone. Proper selection or design of a prosthesis that is appropriately sized and shaped and proper positioning of that prosthesis are important to ensure an optimal surgical outcome. A surgeon may analyze damaged bone to assist with prosthesis selection, design and/or positioning, as well as surgical steps to prepare bone or tissue to receive or interact with a prosthesis. These surgical steps may include removing bone areas (e.g., osteophytes) for proper positioning of the prosthesis.

### SUMMARY

This disclosure describes example techniques for identifying bone areas for removal during a medical procedure. For instance, with the example techniques, a computing system may obtain image data (e.g., 2D or 3D image data) of one or more bones (e.g., humeral head) prior to the medical procedure (e.g., surgery). For example, the image information may be obtained from computed tomography (CT) scans.

Medical personnel (e.g., a surgeon) may use the image data to plan for the medical procedure by identifying one or more bone areas in the image data that may be removed during the medical procedure. For example, the medical personnel may identify one or more osteophytes in the image data that are to be removed in initial steps of surgery before the surgeon may implant a prosthesis. In some examples, the computing system may remove the one or more osteophytes from the image data (e.g., in response to feedback from the medical personnel or without feedback from the medical personnel) to create a virtual model of how a bone should be prepped during surgery for the implant. In some examples, rather than or in addition to the medical personnel identifying the osteophytes, the computing system may be configured to identify the osteophytes.

Osteophytes are provided as one example, and the techniques described in this disclosure should not be considered limited to osteophytes. For instance, the example techniques may be applicable to surgeries that utilize removal of bone or bony structures, such as in patella surgeries. The example techniques may be applicable to shoulder surgeries such as part of Reversed Arthroplasty (RA), Augmented Reverse Arthroplasty (RA), Standard Total Shoulder Arthroplasty (TSA), Augmented Total Shoulder Arthroplasty (TSA), or Hemispherical shoulder surgeries. The example techniques may be used for Bony Increased Offset-Reversed Shoulder Arthroplasty (BIO-RSA™) surgical techniques.

The virtual model (e.g., image data of the bone with one or more bone areas removed) may be used by a surgeon as a guide during the medical procedure. For example, a computing system may overlay the virtual model over the corresponding bone during surgery to help guide the surgeon on what bone areas to remove. In some examples, the computing system may display the virtual model proximate to the corresponding bone during surgery to help guide the surgeon on what bone areas to remove. That is, a surgeon or other medical professional may view the virtual model overlaid on or proximate to the bone during the surgery (e.g., in virtual reality, mixed reality, or augmented reality representation). For instance, in mixed or augmented reality, the surgeon may view the real bone through a screen of goggles for mixed or augmented reality, and the goggles may overlay the virtual model on the real bone on the screen or display the virtual model proximate to the real bone on the screen. In this example, patient bone and tissue are viewable through the screen, and the bone having the osteophytes is also viewable through the screen but with the virtual model overlaid on or proximate to the bone having the osteophytes.

In some examples, the virtual model may be in a first color (e.g., green). The computing device may display an indication with one or more characteristics overlaid on or proximate to the one or more bone areas that have been removed from the virtual model to further indicate/highlight which bone area(s) to remove. “Indication,” as used in this disclosure, may refer to a way in which information is provided to show to a surgeon which bone areas and how much bone areas have been removed or are to be removed. As one example, the “indication” may be a color overlaid on part of the bone that is to be removed, and the indication is updated (e.g., the color is changed) as the bone is being removed.

For example, the computing system may obtain live image data of the bone during surgery and compare that live image data to the virtual model in real-time or near real-time to determine a difference map of the virtual model to the corresponding bone. The computing device may determine the one or more characteristics of the indication (e.g., size, area, volume, shape, colors) overlaid on or proximate to the one or more bone areas based on the difference map. For example, the computing device may initially display the indication over or proximate to the one or more bone areas in a second color. As the surgeon removes one or more portions of the bone area(s), the computing device may update the difference map in real-time or near real-time. In some examples, the indication may include different colors at once to show different amounts of bone material still to be removed.

As the difference between the virtual model and the corresponding bone is reduced (e.g., as a result of the surgeon removing one or more portions of the bone area(s)), the computing device may update the one or more characteristics including the color of the indication. For example, the computing device may change the color of the indication from a first color to a second color and from the second color to a third color as the difference between the virtual model and the corresponding bone is reduced. The computing device may change the color of the indication to a fourth color or remove the indication entirely when the difference between the virtual model and the corresponding bone is below a minimum threshold. In this manner, the example techniques may help the surgeon plan for the medical procedure and help guide the surgeon during the procedure in real-time or near real-time.

The example techniques described in this disclosure provide for a practical application to imaging techniques that improve the overall surgical procedure. For instance, pre-surgery and intra-surgery imaging is registered together so that the intra-surgery imaging can be overlaid over or displayed proximate to the pre-surgery imaging. Then, the computing system updates in real-time or near real-time the imaging resulting in real-time or near real-time information regarding the amount of bone that the surgeon has removed, as well as guidance to the surgeon regarding whether sufficient bone has been removed (e.g., based on the color presented to the surgeon).

In one example, the disclosure describes a method comprising obtaining first image data of an anatomical object before a surgery, removing one or more areas of the anatomical object from the first image data to form a virtual model of the anatomical object before the surgery, obtaining second image data of the anatomical object during the surgery, generating information of the virtual model for overlay on or for displaying proximate to the anatomical object based on the second image data, generating one or more indications overlaid on or proximate to one or more bone areas of the anatomical object corresponding to the one or more areas removed from the first image data, and outputting the information of the virtual model for overlay on or for displaying proximate to the anatomical object and the one or more indications to a user during the surgery.

In one example, the disclosure describes a system comprising memory configured to store first image data of an anatomical object before a surgery and processing circuitry. The processing circuitry is configured to obtain the first image data of the anatomical object before the surgery, remove one or more areas of the anatomical object from the first image data to form a virtual model of the anatomical object before the surgery, obtain second image data of the anatomical object during the surgery, generate information of the virtual model for overlay on or for displaying proximate to the anatomical object in the second image data, generate one or more indications overlaid on or proximate to one or more bone areas of the anatomical object corresponding to the one or more areas removed from the first image data, and output the information of the virtual model for overlay on or for displaying proximate to the anatomical object and the one or more indications to a user during the surgery.

In one example, the disclosure describes a computer-readable storage medium storing instructions thereon that when executed cause one or more processors to obtain first image data of an anatomical object before a surgery, remove one or more areas of the anatomical object from the first image data to form a virtual model of the anatomical object before the surgery, obtain second image data of the anatomical object during the surgery, generate information of the virtual model for overlay on or for displaying proximate to the anatomical object based on the second image data, generate one or more indications overlaid on or proximate to one or more bone areas of the anatomical object corresponding to the one or more areas removed from the first image data, and output the information of the virtual model for overlay on or for displaying proximate to the anatomical object and the one or more indications to a user during the surgery.

In one example, the disclosure describes a system comprising means for obtaining first image data of an anatomical object before a surgery, means for removing one or more areas of the anatomical object from the first image data to form a virtual model of the anatomical object before the surgery, means for obtaining second image data of the anatomical object during the surgery, generating information of the virtual model for overlay on or displaying proximate to the anatomical object based on the second image data, means for generating one or more indications overlaid on or proximate to one or more bone areas of the anatomical object corresponding to the one or more areas removed from the first image data, and means for outputting the information of the virtual model for overlay on or for displaying proximate to the anatomical object and the one or more indications to a user during the surgery.

The details of various examples of the disclosure are set forth in the accompanying drawings and the description below. Various features, objects, and advantages will be apparent from the description, drawings, and claims.

### BRIEF DESCRIPTION OF DRAWINGS

FIG. 1 is a block diagram illustrating an example computing device that may be used to implement the techniques of this disclosure.

FIG. 2 is a block diagram of an orthopedic surgical system that includes a mixed reality (MR) system, according to an example of this disclosure.

FIG. 3 is a schematic representation of a visualization device for use in a mixed reality (MR) system, according to an example of this disclosure.

FIG. 4 is a conceptual diagram illustrating an example image data generated from a medical imaging scan of patient anatomy.

FIG. 5 is a conceptual diagram illustrating example virtual model of patient anatomy with bone areas in the patient anatomy removed.

FIG. 6 is a conceptual diagram illustrating example virtual model of patient anatomy overlaid over the patient anatomy.

FIG. 7 is a conceptual diagram illustrating an example image data generated from a medical imaging scan of patient anatomy.

FIG. 8 is a conceptual diagram illustrating example virtual model of patient anatomy with bone areas in the patient anatomy removed.

FIG. 9 is a conceptual diagram illustrating example virtual model of patient anatomy overlaid over the patient anatomy.

FIG. 10A is a conceptual diagram illustrating an example anatomical object.

FIG. 10B is a conceptual diagram illustrating example virtual model of patient anatomy overlaid over the patient anatomy with an indication over a bone area to be removed.

FIG. 11A is a conceptual diagram illustrating an example anatomical object.

FIG. 11B is a conceptual diagram illustrating example virtual model of patient anatomy overlaid over the patient anatomy with an indication over a bone area to be removed.

FIG. 12A is a conceptual diagram illustrating an example anatomical object.

FIG. 12B is a conceptual diagram illustrating example virtual model of patient anatomy overlaid over the patient anatomy with an indication over a bone area to be removed.

FIG. 13 is a flowchart illustrating an example method of operation in accordance with one or more example techniques described in this disclosure.

### DETAILED DESCRIPTION

This disclosure describes example techniques to identify one or more bone areas for removal during a surgical procedure. A patient may suffer from a disease that causes damage to the patient anatomy, or the patient may suffer an injury that causes damage to the patient anatomy. For shoulders, as an example of patient anatomy, a patient may suffer from primary glenoid humeral osteoarthritis (PGHOA), rotator cuff tear arthropathy (RCTA), instability, massive rotator cuff tear (MRCT), rheumatoid arthritis (RA), post-traumatic arthritis (PTA), osteoarthritis (OA), or acute fracture, as a few examples. The shoulder is one example, and the example techniques may be applicable to other joint surgeries and/or to other patient anatomy, such as the patella, but other examples are possible.

To address the disease or injury, a surgeon performs a surgical procedure. For a shoulder, for example, a surgeon may perform Reversed Arthroplasty (RA), Augmented Reverse Arthroplasty (RA), Standard Total Shoulder Arthroplasty (TA), Augmented Total Shoulder Arthroplasty (TA), or Hemispherical surgeries, as a few examples. One example of the surgical procedure is the Bony Increased Offset-Reversed Shoulder Arthroplasty (BIO-RSA™). There may be benefits for the surgeon to determine, prior to the surgery, characteristics (e.g., size, shape, and/or location) of bone areas (e.g., osteophytes) to be removed from patient anatomy (e.g., from a bone). For instance, determining the characteristics of the bone areas to remove from patient anatomy may aid in prosthesis selection, design and/or positioning, as well as planning of surgical steps to prepare a surface of the damaged bone to receive or interact with a prosthesis. With advance planning, the surgeon can determine, prior to surgery, rather than during surgery, steps to prepare bone or tissue, tools that will be needed, sizes and shapes of the tools, the sizes and shapes of other characteristics of one or more protheses that will be implanted, and the like.

Part of the surgical planning may involve identifying one or more areas for removal from anatomical object (e.g., humeral head as one example). For example, an osteophyte is bone outgrowth that is formed where the bone has been stressed or cartilage has degraded as a result of osteoarthritis, arthritis or rheumatoid arthritis. Osteophytes should be removed on initial step of the surgery before placing the selected implant. To identify one or more areas for removal from anatomical object, medical personnel (e.g., a clinician or surgeon) may take a plurality of scans (e.g., images) of the patient such as computer tomography (CT) scans) to produce image data, such as 3D image information. The CT scans may provide the surgeon with a relatively complete view of the patient anatomy. For instance, the surgeon may view two-dimensional (2D) images or three-dimensional (3D) images of the CT scans.

In general, CT works by passing focused X-rays through the body and measuring the amount of the X-ray energy absorbed. The amount of X-ray energy absorbed by a known slice thickness is proportional to the density of the body tissue. By taking many such measurements from many angles, the tissue densities can be composed as a cross-sectional image using a computing device. The computing device may generate a grey scale image where the tissue density is indicated by shades of grey.

A computing system may segment the various anatomical objects. Segmenting the anatomical objects refers to information indicating boundaries between anatomical objects (e.g., boundary of the humeral head and the glenoid, as one example). Segmentation may be one way in which to separately view, for different angles, each of the anatomical objects (e.g., view the humeral head without viewing the scapula or other shoulder components). There may be various segmentation techniques. One way to perform segmentation is using the BLUEPRINT™ system available from Wright Medical Technology, Inc. However, there are other ways in which segmentation is performed and the techniques are not limited to any specific way in which to perform segmentation.

From the image of the segmented anatomical object, the surgeon may be able to view not only the bone but osteophytes of the bone. Osteophytes are bony projections from the bone and tend to form along joint margins (e.g., at the end of the humeral head). As used in this disclosure, osteophytes are referred to as the bony projections that developed along the bone. In some examples, osteophytes are bony growths that are the result of damage to the bone, and although bony projections are from the bone, are not part of the healthy bone. In other words, osteophytes are described as bony projections that may be removed during surgery. There may be various causes for the osteophytes such as due to joint damage (e.g., from osteoarthritis).

In one or more examples, a surgeon may desire to remove the osteophytes such as to prepare the bone for an implant. For instance, the osteophytes may block insertion of the implant at a particularly desired location or require insertion of the implant at a less desirable angle. In some cases, even if the osteophytes are not limiting the insertion of the implant (e.g., location or angle), the osteophytes may limit the range of motion. As one example, the osteophytes may limit how much the shoulder of the patient can rotate before an osteophyte interferes with the rotation. The patient may experience similar range of motion limits or other discomfort from osteophytes where the osteophytes are on the knee, spine, hip, ankles, and other bones.

Simply for ease of description, the example techniques are described with respect to the humeral head and osteophytes located on the humeral head. However, the example techniques should not be considered so limited. The example techniques may be extended to other bones as well such as the ankle, hip, patella, etc. In general, as described in more detail, the example techniques may be applicable to surgeries where a virtual model is overlaid on an anatomical object, and where there may be real-time or near real-time updates to the virtual model during surgery. For example, the example techniques may be applicable to surgeries via mixed reality (MR) such that a user (e.g., surgeon) is able to view the virtual model within a real-world scene including real-world anatomical objects (e.g., via an MR head mounted display).

A surgeon may desire to remove the osteophytes from the bone. However, there may be issues in ensuring that osteophytes are noticeable during surgery (e.g., intra-operatively) and then determining during surgery whether the osteophyte removal is sufficient or not.

In some examples, prior to surgery, medical personnel (e.g., surgeon) may identify one or more areas of an anatomical object (e.g., bone areas or osteophytes) to remove from the image data of the anatomical object. Rather than or in addition to a medical personnel, in some examples, it may be possible for the computing system that performed the segmentation to also identify osteophytes (e.g., based on determining what bone without osteophytes should be shaped like for the patient and determining a difference between the image data of the anatomical object and the determined bone shape). In some examples, the medical personnel may need to confirm the osteophytes determined by the computing system are actual osteophytes.

According to techniques in this disclosure, after the medical personnel or the computing system identify, prior to surgery, one or more areas of the anatomical object (e.g., bone areas or osteophytes), the computing system may remove from the image data the identified areas to form a virtual model of how the anatomical object should be prepared for a medical procedure. For example, the medical personnel may identify or highlight (e.g., with a stylus, mouse, touchscreen or any other input device) one or more areas (or volumes) in the image data that are to be removed in initial steps of surgery before the surgeon may implant a prosthesis. The computing system may remove these identified areas (or volumes) from the image data to form a virtual model of how the bone should be shaped before the implant is placed.

A surgeon may use the virtual model for guidance on which one or more areas to remove of an anatomical object during surgery. For example, a computing system may overlay the virtual model over the anatomical object during surgery (e.g., in mixed or augmented reality representation in a head-mounted display or any other display). As another example, the computing system may display the virtual model proximate to the anatomical object during surgery. The virtual model may guide the surgeon with where to remove osteophytes so that the surgeon can understand/visualize the end result of the having bone with osteophytes removed.

In this way, the example techniques may be used to identify or highlight the one or more areas of the anatomical object to remove in the initial steps of the surgical procedure. The example techniques provide a practical application whereby the example techniques generate information indicative of the shape, size and/or location of the one or more areas (or volumes) to remove during the surgical procedure. Using this information, the surgeon can better plan and execute a repair or replacement surgery to address the patient injury or disease.

As described in more detail, in one or more examples, a computing system may obtain image data of an anatomical object to determine the size, shape, and/or location of areas (or volumes) to remove. As one example, the image data may be a 3D volume represented by a graphical shape volume that the computing system defines as a point cloud. As one example, the point cloud may be vertices of a plurality of primitives (e.g., triangles) interconnected together and rasterized to form the shape. For instance, the computing system may define the vertices of the primitives and define interconnection of vertices of a primitive with vertices of other primitives to form a virtual model of the anatomical object. A point cloud being vertices of primitives is merely one example and should not be considered limiting.

Also, the image data being a 3D volume is merely one example. In some examples, the image data may be a 2D surface (e.g., 2D contour or 2D wireframe), and the example techniques may be applied to image information from 2D image slices. The result of the example techniques described in the disclosure on the 2D image slices may be a series of 2D shapes that represent the anatomical object. The series of 2D shapes may then be combined to form a 3D representation of the anatomical object. For ease, this disclosure describes techniques for a 3D volume, but the techniques are not so limited.

As described above, the virtual model (e.g., image data of the bone with one or more bone areas or volumes removed) may be used by a surgeon as a guide during the medical procedure. For example, a computing system may overlay (e.g., superimpose) the virtual model over the corresponding bone or display the virtual model proximate to the corresponding bone (e.g., less than half-meter from the corresponding bone) during surgery to help guide the surgeon on what bone areas to remove. That is, a surgeon or other medical professional may view the virtual model overlaid on or proximate to the bone during the surgery (e.g., in mixed or augmented reality representation). In mixed or augmented reality, the actual bone is viewable through the screen, and the virtual model is overlaid on top of the bone or displayed proximate to the bone, so that on the screen the virtual model appears to be overlaid on top of the bone or proximate to the bone. In some examples, the virtual model may be in a first color. The computing system may display an indication with one or more characteristics overlaid on or proximate to the one or more bone areas that have been removed from the virtual model to further indicate/highlight which bone area(s) to remove, as described below. The “indication” may refer to information that is displayed that shows how much of the bone has been removed, how much of the bone is to be removed, or some other information indicating how bone removal is progressing. The indication may be displayed (e.g., such as different colors) or may be provided in some other way (e.g., haptic or audible information).

A computing system may obtain live image data of the anatomical object during surgery and compare that live image data to the virtual model in real-time or near real-time to determine a difference map of the virtual model to the corresponding bone. For example, the computing system may determine the distance (e.g., Euclidian distance) from an origin point of a normal vector projected from the virtual model to a terminal point on the surface of the anatomical object in the live image data during the surgical procedure. The computing system may determine this distance from each point of the virtual model corresponding to the one or more areas that were removed by the medical personal to the corresponding points in the anatomical object to determine a difference map for the one or more areas or volumes to be removed. The computing system may then determine the one or more characteristics of the indication (e.g., size, area, volume, shape, colors) overlaid on or proximate to the one or more bone areas based on this difference map. For example, the color of the indication may depend of the difference map and the computing system may initially display the indication over or proximate to the one or more bone areas in red. For example, a first color may correspond to distances above a first threshold, second color may correspond to distances above a second threshold (above the first threshold), and so on. As the surgeon removes one or more portions of the bone area(s), the computing system may update the difference map and the indication in real-time or near real-time to other colors (e.g., red to orange to yellow to green, or any other color combinations). In some examples, an indication may include one or more colors to show the amount of bone material that should be removed.

In addition to or instead of using colors to show the amount of bone material that should removed, the computing system may utilize other techniques. For example, a visualization device worn by the surgeon may provide a haptic response or an audio response, as two examples. For ease, the disclosure is described with respect to visual response regarding how much bone material should be removed, but the example techniques should not be considered so limited.

A surgeon may remove one or more portions of one or more bone areas (or volumes) from the anatomical object during surgery. The computing system may continue to obtain live image data of the anatomical object during the surgery, update the difference map (e.g., determine differences between points in the virtual model to points in the live image data), and update the indication based on the updated difference map. In this way, the surgeon may know what and the amount of bone areas to remove.

Moreover, the example techniques may be utilized as part of robot assisted surgery, also referred to as robot guided surgery. For example, a robot configured to assist with surgery may receive information of the virtual model (e.g., coordinates of the vertices of polygons used to form the virtual model). In some examples, similar to how the computing system may register the virtual model to the anatomical object, the robot may register the virtual model to the anatomical object based on image data captured during surgery. Based on the registration of the virtual model, the robot may assist in limiting the ability of the surgeon to remove bone that should not be removed. That is, the robot may utilize the virtual model as a guide, and if the surgeon is removing bone that would cause the final bone to not be like the virtual model, the robot may assist in limiting the ability of the surgeon to remove bone that should not be removed.

For example, the robot may hold a surgical tool, and the surgeon may also hold the surgical tool. During the surgery, the surgeon can move the surgical tool, while the robot holds the tool. If the surgeon attempts to remove bone that should not be removed (e.g., as determined by the robot based on the virtual model), the robot may provide an indication to the surgeon that he or she is attempting to remove bone that should not be removed. For instance, the robot may provide initial resistance that restricts the ease at which the surgeon can move the surgical tool. The robot may output haptic feedback, an audible tone, a visual indication, or some form of output that notifies the surgeon that he or she may be attempting to remove bone that should not be removed. The surgeon may have the option of overriding the robot if needed so that the surgeon can freely perform the surgery. In some examples, the surgeon may update the virtual model or parameters that the robot uses to determine if the surgeon is removing bone that should not be removed.

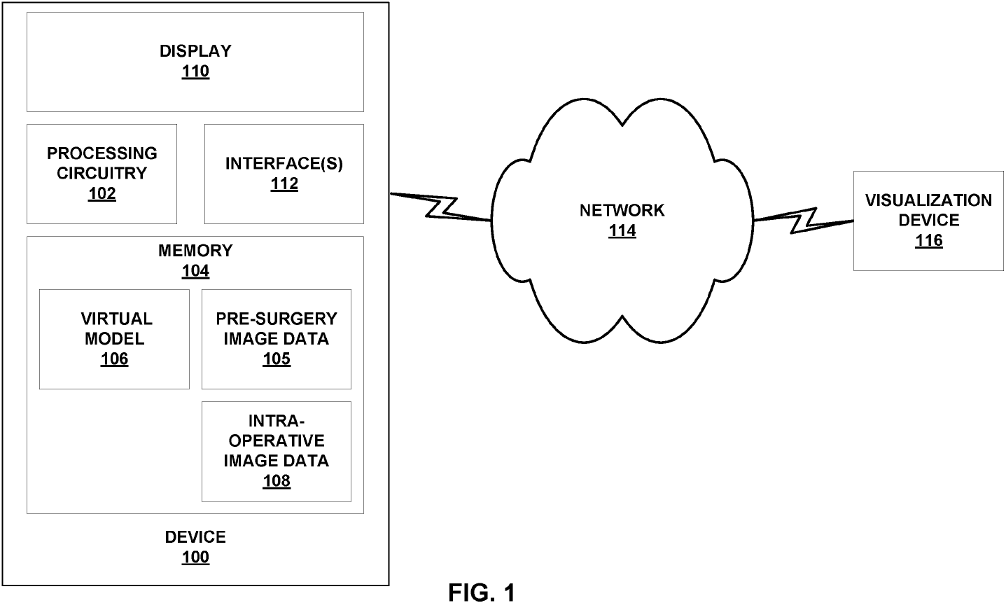

FIG. 1 is a block diagram illustrating an example computing system that may be used to implement the techniques of this disclosure. FIG. 1 illustrates device 100, which is an example of a computing device configured to perform one or more example techniques described in this disclosure.

Device 100 may include various types of computing devices, such as server computers, personal computers, smartphones, laptop computers, and other types of computing devices. Device 100 includes processing circuitry 102, memory 104, display 110, and image capture devices 113. Display 110 is optional, such as in examples where device 100 is a server computer.

Examples of processing circuitry 102 include one or more microprocessors, digital signal processors (DSPs), application specific integrated circuits (ASICs), field programmable gate arrays (FPGAs), discrete logic, software, hardware, firmware or any combinations thereof. In general, processing circuitry 102 may be implemented as fixed-function circuits, programmable circuits, or a combination thereof. Fixed-function circuits refer to circuits that provide particular functionality and are preset on the operations that can be performed. Programmable circuits refer to circuits that can programmed to perform various tasks and provide flexible functionality in the operations that can be performed. For instance, programmable circuits may execute software or firmware that cause the programmable circuits to operate in the manner defined by instructions of the software or firmware. Fixed-function circuits may execute software instructions (e.g., to receive parameters or output parameters), but the types of operations that the fixed-function circuits perform are generally immutable. In some examples, the one or more of the units may be distinct circuit blocks (fixed-function or programmable), and in some examples, the one or more units may be integrated circuits.

Processing circuitry 102 may include arithmetic logic units (ALUs), elementary function units (EFUs), digital circuits, analog circuits, and/or programmable cores, formed from programmable circuits. In examples where the operations of processing circuitry 102 are performed using software executed by the programmable circuits, memory 104 may store the object code of the software that processing circuitry 102 receives and executes, or another memory within processing circuitry 102 (not shown) may store such instructions. Examples of the software include software designed for surgical planning.

Although processing circuitry 102 is illustrated as being within device 100, the example techniques are not so limited. In some examples, processing circuitry 102 represents the circuitry in different devices that are used together to perform the example techniques described in this disclosure. Therefore, processing circuitry 102 may be configured as processing circuitry of a system configured to perform the example techniques described in this disclosure. That is, processing circuitry 102 may be distributed processing circuitry across different devices. However, in some examples, processing circuitry 102 may be processing circuitry that is local to device 100. In this disclosure, when the description is described with respect to processing circuitry 102, such description includes examples where processing circuitry 102 is local to device 100 or where processing circuitry 102 is distributed circuitry across different devices (e.g., servers).

Memory 104 may be formed by any of a variety of memory devices, such as dynamic random access memory (DRAM), including synchronous DRAM (SDRAM), magnetoresistive RAM (MRAM), resistive RAM (RRAM), or other types of memory devices. Examples of display 110 include a liquid crystal display (LCD), a plasma display, an organic light emitting diode (OLED) display, or another type of display device.

Similar to processing circuitry 102, although memory 104 is illustrated as being local to device 100, the techniques are not so limited. In some examples, memory 104 may be distributed across various devices. In this way, processing circuitry 102 and memory 104 provide for a way to perform distributed operations across different devices in some examples. In some examples, processing circuitry 102 and memory 104 may be local to device 100, and the computations and storage may be performed local to device 100. Different permutation and combinations are possible (e.g., memory 104 is local to device 100 and processing circuitry 102 is distributed, processing circuitry 102 is local to device 100 and memory 104 is distributed, processing circuitry 102 and memory 104 are both local to device 100, or processing circuitry 102 and memory 104 are both distributed).

Device 100 may include interfaces 112 that allow device 100 to receive input data and instructions from one or more input devices (e.g., a mouse, stylus, keyboard, touchscreen, or any other input device) and output data and instructions to output devices. In some examples, interfaces 112 may include hardware circuitry that enables device 100 to communicate (e.g., wirelessly or using wires) to other computing systems and devices, such as visualization device 116. Network 114 may include various types of communication networks including one or more wide-area networks, such as the Internet, local area networks, and so on. In some examples, network 114 may include wired and/or wireless communication links. In some examples, device 100 may use interfaces 112 receive data and instructions from visualization device 116 via network 114.

Visualization device 116 may utilize various visualization techniques to display image content to a surgeon. Visualization device 116 may be a mixed reality (MR) visualization device, augmented reality (AR) visualization device, holographic projector, or other device for presenting extended reality (XR) visualizations. In some examples, visualization device 116 may be a Microsoft HOLOLENS™ headset, available from Microsoft Corporation, of Redmond, Washington, USA, or a similar device, such as, for example, a similar MR visualization device that includes waveguides. The HOLOLENS™ device can be used to present 3D virtual objects via holographic lenses, or waveguides, while permitting a user to view actual objects in a real-world scene, i.e., in a real-world environment, through the holographic lenses.

Visualization device 116 may utilize visualization tools that are available to utilize patient image data to generate three-dimensional models of bone contours to facilitate preoperative planning for joint repairs and replacements. These tools allow surgeons to design and/or select surgical guides and implant components that closely match the patient’s anatomy. These tools can improve surgical outcomes by customizing a surgical plan for each patient. An example of such a visualization tool is the BLUEPRINT™ system available from Wright Medical Technology, Inc. The BLUEPRINT™ system provides the surgeon with two-dimensional planar views of the bone repair region as well as a three-dimensional virtual model of the repair region. The surgeon can use the BLUEPRINT™ system to select, design or modify appropriate implant components, determine how best to position and orient the implant components and how to shape the surface of the bone to receive the components, and design, select or modify surgical guide tool(s) or instruments to carry out the surgical plan. The information generated by the BLUEPRINT™ system is compiled in a preoperative surgical plan for the patient that is stored in a database at an appropriate location (e.g., on a server in a wide area network, a local area network, or a global network) where it can be accessed by the surgeon or other care provider, including before and during the actual surgery.

As illustrated, memory 104 stores pre-surgery image data 105, virtual model 106, and intra-operative image data 108. Pre-surgery image data 105 refers to image data of an anatomical object before a surgery. For instance, pre-surgery image data 105 includes image data of the bone and the osteophytes attached to the bone. Pre-surgery image data 105 may be captured via a CT scan prior to surgery.

In some examples, display 110 may display pre-surgery image data 105 to the medical personnel, and the medical personnel may mark areas of the anatomical object that are to be removed. For example, the medical personnel may utilize a stylus to trace the osteophytes (e.g., areas of the anatomical object) that are to be removed from the bone (e.g., anatomical object).

In some examples, processing circuitry 102 may identify the osteophytes. For instance, using statistical shape model (SSM) techniques, processing circuitry 102 may determine how the bone should be shaped for the patient without the osteophytes. Processing circuitry 102 may determine a difference in the bone shape determined from the SSM techniques and pre-surgery image data 105 to determine the osteophytes.

As one example of an SSM technique, the SSM may be a shape model of how a statistically mean bone appears. The SSM may represent “healthy” bone without the osteophytes. Processing circuitry 102 may perform image processing such as alignment of coordinates of the bone in image data 105 to the SSM. The alignment may be based on key marker points on the bone in image data 105 and the SSM. Processing circuitry 102 may then morph, scale, etc. either or both the bone in image data 105 or the SSM until the SSM and the SSM and the bone in the image data 105 register. The registration may include adjusting the size and shape of the SSM to the bone in image data 105 and adjust a location of the SSM to align with the bone in image data 105. The result of the registration may be a shape model that is an approximation of the bone in image data 105 without the osteophytes.

In this way, as one example, processing circuitry 102 may determine a shape of the bone without the one or more osteophytes. Processing circuitry 102 may identify the one or more osteophytes based on a difference between image data 105 (e.g., bone in image data 105) and the determined shape of the bone without the one or more osteophytes.

In both examples, after determining the osteophytes, processing circuitry 102 may remove one or more areas (e.g., determined osteophytes) of the anatomical object from pre-surgery image data 105 to form virtual model 106 of the anatomical object before the surgery. That is, virtual model 106 is an example of image data that is stored after processing circuitry 102 removes bone areas from an anatomical object from pre-surgery image data.

Virtual model 106 may be a graphical surface image that defines a 3D volume corresponding to an anatomical object. Virtual model 106 may be is defined as 3D point cloud. As one example, the 3D point cloud may define a plurality of interconnected primitives (e.g., triangles) to form a particular shape. Virtual model 106 may be a three-dimensional (3D) shape, but the example techniques described in this disclosure may be applicable to examples where virtual model 106 is a two-dimensional (2D) shape (e.g., 2D contour or 2D wireframe) as well. Although one virtual model 106 is illustrated, memory 104 may store a plurality of virtual models.

In examples described in this disclosure, when memory 104 or computing device 100 is described as storing virtual model 106, memory 104 or computing device 100 may be considered as storing information from which processing circuitry 102 can graphically construct virtual model 106 (e.g., by rendering the shape directly using point information or indirectly using parametric information). As one example, virtual model 106 may include coordinates for the vertices of the primitives that form virtual model 106 and interconnection of the primitives. In some examples, operations on virtual model 106 may be considered as operations on the point cloud that used to form virtual model 106 such as vertices of primitives that form virtual model 106. Virtual model 106 may be defined using implicit representations as well (e.g., based on equations defining the shape).

In some examples, virtual model 106 defines a closed surface. As described above, a closed surface is a delimitation of a subset of the Euclidian space being closed (that is, containing all its limit points) and bounded (that is, having all its points lie within some fixed distance of each other). For instance, a closed surface is a surface where two levels of derivative can be applied on any point. As one example, a closed surface contains a volume of space that is enclosed from all directions. A sphere is one example of a closed surface because it contains a volume of space that is enclosed from all directions. There may be other examples of closed surface (e.g., cubes, pyramids, etc. are examples of closed surfaces).

In some examples, virtual model 106 may be received from another computing device prior to surgery. In some examples, virtual model 106 may be created with device 100 prior to surgery. As described above, device 100 may receive CT scans from another computing device. Device 100 may display the image data on display 110 and medical person may select or highlight one or more areas to remove in the image data (e.g., with a stylus, mouse, touchscreen or any other input device). Processing circuitry 102 may remove the one or more areas (or volumes) of the anatomical object in the image data to generate virtual model 106 prior to surgery. In this way, the surgeon or medical personnel may plan for the surgery accordingly. For example, the patient may have an injured shoulder requiring surgery, and for the surgery or possibly as part of the diagnosis, the surgeon may use virtual model 106 to plan the surgery.

In one or more examples, image data 108 may be live image scans of anatomy of the patient during the surgery, which may be stored as 2D image information or 3D volumetric image information. For example, the surgeon may wear visualization device 116 during the surgery, and image data 108 may be the result of image data captured by visualization device 116.

Processing circuitry 102 may register (e.g., align) virtual model 106 to the corresponding anatomical object in intra-operative image data 108 in real-time or near real-time. For example, using depth data, processing circuitry 102 may determine the distance between virtual model 106 and the real bone. Processing circuitry 102 may perform such comparison triangle-by-triangle between a mesh of triangles that makes up the virtual model and the mesh of triangles that make up the real bone in image data 108 (e.g., such as by computing the distance map between the two meshes). Processing circuitry 102 may utilize an iterative closest point (ICP) algorithm to adjust virtual model 106 until virtual model 106 is registered with the image data corresponding to the real anatomical object in image data 108. Then, when displayed, processing circuitry 102 may overlay virtual model 106 over the corresponding anatomical object in intra-operative image data 108. In some examples, processing circuitry 102 may add an offset to the coordinates of virtual model 106 so that, when displayed, virtual model 106 appears proximate but not necessarily on top of the bone.

Processing circuitry 102 may then compare the anatomical object in intra-operative image data 108 to virtual model 106 in real-time or near real-time to determine a difference map of virtual model 106 to the corresponding anatomical object. Processing circuitry 102 may then determine one or more characteristics (e.g., size, area, volume, shape, colors) of one or more indications to be overlaid on or displayed proximate to one or more bone areas (or volumes) of the anatomical object that were removed from virtual model 106. According to examples of this disclosure, processing circuitry 102 may overlay virtual model 106 or display virtual model 106 proximate to the one or more indications over the anatomical object in intra-operative image data 108 and interface 112 may output intra-operative image data 108 with the virtual model 106 and the one or more indications to visualization device 116 via network 114. The one or more indications may also be proximate to the anatomical object in the intra-operative image data 108.

Accordingly, the bone is visible through the screen for visualization device 116. In some examples, virtual model 106 and the one or more indications are displayed on the screen of visualization device 116 as being overlaid on the bone. In some examples, virtual model 106 is displayed on the screen of visualization device 116 as being proximate to the bone, and the one or more indications are displayed as overlaid on the bone. In some examples, virtual model 106 is displayed on the screen of visualization device 116 as overlaid on the bone, and the one or more indications are displayed as being proximate to the bone. In some examples, virtual model 106 and the one or more indications are displayed on the screen of visualization device 116 as being proximate to the bone. It may be possible that the one or more indications are overlaid on virtual model 106 in examples where both virtual model 106 and the one or more indications are displayed on the screen of visualization device 116 as being proximate to the bone.

Initially the one or more bone areas in intra-operative image data 108 may be identical to the one or more areas that processing circuitry 102 removed from pre-surgery image data 105 to form virtual model 106. That is, initially, intra-operative image data 108 should look the same as virtual model 106 with the osteophytes. Virtual model 106 may the idealized final result of how the bone should appear once the osteophytes are removed. Then, during surgery, as the surgeon removes the osteophytes (e.g., by shaving them surgically), intra-operative image data 108 will reflect the osteophytes as they are being removed. Processing circuitry 102 may repeatedly determine difference in intra-operative image data 108 and virtual model 106 to determine whether the surgeon has sufficiently removed the osteophyte. Processing circuitry 102 may provide indications, intra-operatively, to the surgeon via visualization device 116 indicating whether the surgeon has sufficiently removed the osteophyte.

Intra-operative image data 108 is the image data that processing circuitry 102 uses to determine the surgeon has sufficiently removed the osteophyte. However, processing circuitry 102 may not cause visualization device 116 to display intra-operative image data 108. Rather, the real-life bone that is being shaved to remove osteophytes is visible to intra-operatively to the surgeon through the screen of visualization device 116. In other words, intra-operative image data 108 is the image data used for processing but displaying of intra-operative image data 108 may not be needed since the bone is intra-operatively viewable through the screen of visualization device 116. In some examples, it may be possible to present on the screen of visualization device 116 the intra-operative image data 108 (e.g., for confirmation of accuracy of virtual model 106, or to assist the surgeon is viewing the bone from different angles without needing to move the patient, etc.). However, presenting intra-operative image data 108 on the screen may not be necessary.

In some examples, the surgeon may be able to wear visualization device 116 during surgery to generate intra-operative image data 108 of an anatomical object with virtual model 106 overlaid on the anatomical object in real-time or near real-time (e.g., in augmented or mixed reality). In some examples, the surgeon may be able to view one or more indications that indicate whether the surgeon has sufficiently removed the osteophytes (e.g., a first color to indicate that a lot of the osteophyte needs to be removed, a second color to indicate that most of the osteophyte has been removed, and a third color to indicate that the osteophyte is removed). In some examples, the one or more indications may be overlaid on or may be proximate to virtual model 106. As described in more detail below, the comparison between the anatomical object in intra-operative image data 108 and virtual object 106 may be an iterative operation where processing circuitry 102 determines points on virtual model 106 and normal vectors from those points on virtual model 106 to the anatomical object intra-operative image data 108 to determine the difference map, and update the one or more indications based on that difference map. For example, the color of the one or more indications may depend of the difference map. As the surgeon removes one or more portions of the bone area(s), processing circuitry 102 may update the difference map and the indications in real-time or near real-time. In this way, the surgeon may be able to use the virtual model and the one or more indications as a guide for removing the one or more bone areas during surgery.

Accordingly, displaying virtual model 106 after osteophytes removal will allow the surgeon to understand/visualize the result the surgeon is trying to achieve. Virtual model 106 may be an overlay to the real bone but virtual model 106 can also be displayed just close (e.g. proximate) to the real bone as a visual help. The same may be for the one or more indications. For example, the screen of visualization device 116 may reproduce a virtual scene with virtual model 106, the indication (with colors) but not overlay over the bone to ensure the scene is clear for the surgeon; however, overlaying virtual model 106 and/or the indications over the bone may be possible. In some examples, there may be tracker tool showing on the virtual scene where the tool is and if the tool corresponds to a part that needs to be removed (e.g., the tool is over the osteophyte).

As described above, the example techniques may be utilized as part of robot assisted surgery, also referred to as robot guided surgery. For example, a robot configured to assist with surgery may receive virtual model 106, and in some examples intra-operative image data 108. The robot may utilize virtual model 106 and determine the location of virtual model 106 in the real world based on intra-operative image data 108. For instance, the robot may similarly register virtual model 106 to anatomical object, or may utilize intra-operative image data 108 to determine the location of virtual model 106. Based on virtual model 106, the robot may form a guide that defines contours of areas from where the surgeon can remove bone and areas from where the surgeon may not or should not remove bone. In some examples, the surgeon may input the contour of areas from where the surgeon can remove bone and areas from where the surgeon may not or should not remove bone.

During surgery, the robot is configured to hold a surgical tool, and the surgeon moves the surgical tool during the surgery while the robot holds the surgical tool. In most cases, the robot may allow the surgeon to move the surgical tool freely, so long as the surgical tool remains within the areas where bone can be removed. If the robot determines that the surgeon is moving the surgical tool and removing bone in areas from where the surgeon may not remove bone, the robot may provide an indication to the surgeon that he or she is attempting to remove bone that should not be removed. For instance, the robot may provide initial resistance that restricts the ease at which the surgeon can move the surgical tool. The robot may output haptic feedback, an audible tone, a visual indication, or some form of output that notifies the surgeon that he or she may be attempting to remove bone that should not be removed. The surgeon may have the option of overriding the robot if needed so that the surgeon can freely perform the surgery. In some examples, the surgeon may update parameters that the robot uses to determine if the surgeon is removing bone that should not be removed.

In some examples, the example techniques may be applied to specific anatomical objects such as humeral head or specific soft tissue. However, the example techniques are not so limited. The example techniques may be performed with various types of anatomical objects. Examples of the anatomical objects include the coracoid process, acromion, diaphysis, condyles, capitellum, trochlea, clavicle, femur, tibia, patella, fibula, calcaneus, talus, and navicular.

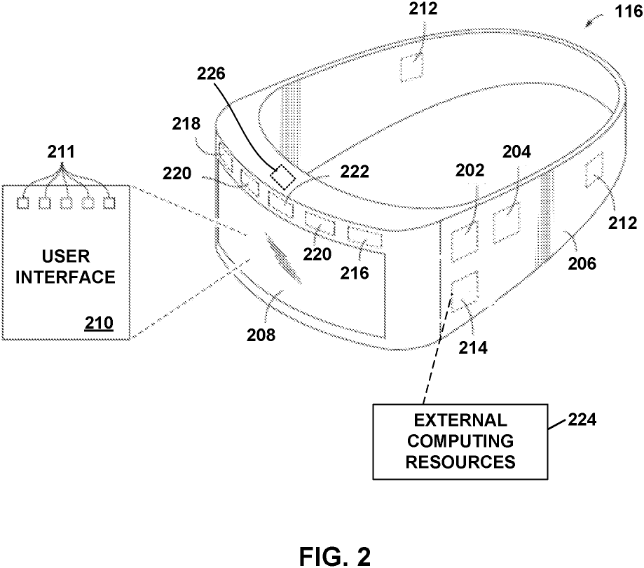

FIG. 2 is a schematic representation of visualization device 116 for use in a system, such as the system of FIG. 1, according to an example of this disclosure. As shown in the example of FIG. 2, visualization device 116 can include a variety of electronic components found in a computing system, including one or more processor(s) 202 (e.g., microprocessors or other types of processing units) and memory 204 that may be mounted on or within a frame 206. Furthermore, in the example of FIG. 2, visualization device 116 may include a transparent screen 208 that is positioned at eye level when visualization device 116 is worn by a user. In some examples, screen 208 can include one or more liquid crystal displays (LCDs) or other types of display screens on which images are perceptible to a surgeon who is wearing or otherwise using visualization device 116 via screen 208. Other display examples include organic light emitting diode (OLED) displays. In some examples, visualization device 116 can operate to project 3D images onto the user’s retinas.

In some examples, screen 208 may include see-through holographic lenses. sometimes referred to as waveguides, that permit a user to see real-world objects through (e.g., beyond) the lenses and also see holographic imagery projected into the lenses and onto the user’s retinas by displays, such as liquid crystal on silicon (LCoS) display devices, which are sometimes referred to as light engines or projectors, operating as an example of a holographic projection system 226 within visualization device 116. In other words, visualization device 116 may include one or more see-through holographic lenses to present virtual images to a user. Hence, in some examples, visualization device 116 can operate to project 3D images onto the user’s retinas via screen 208, e.g., formed by holographic lenses. In this manner, visualization device 116 may be configured to present a 3D virtual image to a user within a real-world view observed through screen 208, e.g., such that the virtual image appears to form part of the real-world environment. In some examples, visualization device 208 may be a Microsoft HOLOLENS™ headset, available from Microsoft Corporation, of Redmond, Washington, USA, or a similar device, such as, for example, a similar MR visualization device that includes waveguides. The HOLOLENS™ device can be used to present 3D virtual objects via holographic lenses, or waveguides, while permitting a user to view actual objects in a real-world scene, i.e., in a real-world environment, through the holographic lenses.

Although the example of FIG. 2 illustrates visualization device 116 as a head-wearable device, visualization device 116 may have other forms and form factors. For instance, in some examples, visualization device 116 may be a handheld smartphone or tablet.

Visualization device 116 can also generate a user interface (UI) 210 that is visible to the user, e.g., as holographic imagery projected into see-through holographic lenses as described above. For example, UI 210 can include a variety of selectable widgets 211 that allow the user to interact with a mixed reality (MR) system. Imagery presented by visualization device 116 may include, for example, one or more 3D virtual objects. Visualization device 116 also can include a speaker or other sensory devices 212 that may be positioned adjacent the user’s ears. Sensory devices 212 can convey audible information or other perceptible information (e.g., vibrations) to assist the user of visualization device 116.

Visualization device 116 can also include a transceiver 214 to connect visualization device 116 to network 114 and/or to a computing cloud, such as via a wired communication protocol or a wireless protocol, e.g., Wi-Fi, Bluetooth, etc. Visualization device 116 also includes a variety of sensors to collect sensor data, such as one or more optical camera(s) 216 (or other optical sensors) and one or more depth camera(s) 222 (or other depth sensors), mounted to, on or within frame 206. In some examples, the optical sensor(s) 216 are operable to scan the geometry of the physical environment in which a user is located (e.g., an operating room) and collect two-dimensional (2D) optical image data (either monochrome or color). Depth sensor(s) 222 are operable to provide 3D image data, such as by employing time of flight, stereo or other known or future-developed techniques for determining depth and thereby generating image data in three dimensions. Other sensors can include motion sensors 220 (e.g., Inertial Mass Unit (IMU) sensors, accelerometers, etc.) to assist with tracking movement.

Visualization device 116 may include one or more processors 202 and memory 204 (e.g., within frame 206 of the visualization device 116). In some examples, one or more external computing resources 224 (e.g., processing circuitry 102 and memory 104 of FIG. 1) process and store information, such as sensor data, instead of or in addition to in-frame processor(s) 202 and memory 204. In this way, data processing and storage may be performed by one or more processors 202 and memory 204 within visualization device 116 and/or some of the processing and storage requirements may be offloaded from visualization device 116. Hence, in some examples, one or more processors that control the operation of visualization device 116 may be within visualization device 116 (e.g., as processor(s) 202, also called processing circuitry 202). Alternatively, in some examples, at least one of the processors that controls the operation of visualization device 116 may be external to visualization device 116 (e.g., as processing circuitry 102). Likewise, operation of visualization device 116 may, in some examples, be controlled in part by a combination one or more processors 202 within the visualization device and processing circuitry 102 external to visualization device 116. Accordingly, in some examples, the processing circuitry configured to perform one or more examples described in this disclosure may include processing circuitry 102, one or more processors 202, or a combination of processing circuitry 102 and one or more processors 202.

In one or more examples described in this disclosure, one or more processors 202 may cause transceiver 214 to transmit the image data that is captured by the optical cameras 216 or optical cameras 216 and depth cameras 222 to processing circuitry 102, which stores the image data as intra-operative image data 108. Transceiver 214 may receive information indicative of virtual model 106 and one or more processors 202 may cause screen 208 to display virtual model 106 overlaid on the actual bone of the patient. In some examples, screen 208 may display virtual model 106 overlaid on image data 108, but this may not be required in all examples. In addition, transceiver 214 may receive from processing circuitry 102 information for one or more indications of bone areas of the anatomical object corresponding to the one or more areas removed from the pre-surgery image data 105.

For example, the one or more areas removed from pre-surgery image data 105 refers to the osteophytes that processing circuitry 102 removed from pre-surgery image data 105 to form virtual model 106. Indications of the bone areas of the anatomical object corresponding to the one or more areas removed from pre-surgery image data 105 may refer to indication of how much of osteophytes have actually been removed during surgery or how close the surgeon is to removing the osteophytes. Processing circuitry 102 may determine the indications of the bone area based on a comparison, in real-time or near real-time, between virtual model 106 and intra-operative image data 108. Processing circuitry 102 may overlay the indications of the bone areas on top of the actual bone or display the indications of the bone areas proximate to the actual bone, which is then presented to the surgeon.

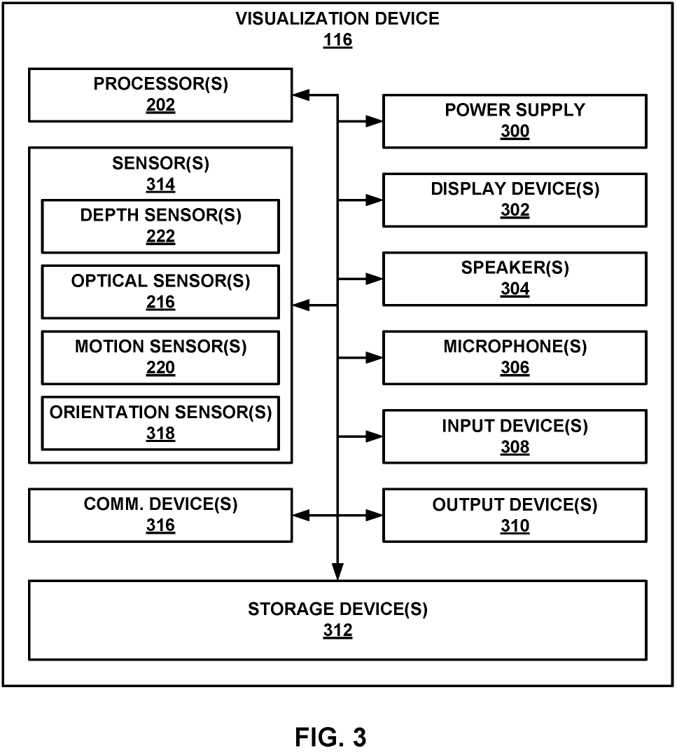

FIG. 3 is a block diagram illustrating example components of visualization device 116. In the example of FIG. 3, visualization device 116 includes processors 202, a power supply 300, display device(s) 302, speakers 304, microphone(s) 306, input device(s) 308, output device(s) 310, storage device(s) 312, sensor(s) 314, and communication devices 316. In the example of FIG. 3, sensor(s) 316 may include depth sensor(s) 222, optical sensor(s) 216, motion sensor(s) 220, and orientation sensor(s) 318. Optical sensor(s) 216 may include cameras, such as Red-Green-Blue (RGB) video cameras, infrared cameras, or other types of sensors that form images from light. Display device(s) 302 (e.g., screen 208) may display imagery to present a user interface to the user.

Speakers 304, in some examples, may form part of sensory devices 212 shown in FIG. 2. In some examples, display devices 302 may include screen 208 shown in FIG. 2. For example, as discussed with reference to FIG. 2, display device(s) 302 may include see-through holographic lenses, in combination with projectors, that permit a user to see real-world objects, in a real-world environment, through the lenses, and also see virtual 3D holographic imagery projected into the lenses and onto the user’s retinas, e.g., by a holographic projection system. In this example, virtual 3D holographic objects may appear to be placed within the real-world environment. In some examples, display devices 302 include one or more display screens, such as LCD display screens, OLED display screens, and so on. The user interface may present virtual images of details of the virtual surgical plan for a particular patient.

In some examples, a user may interact with and control visualization device 116 in a variety of ways. For example, microphones 306, and associated speech recognition processing circuitry or software, may recognize voice commands spoken by the user and, in response, perform any of a variety of operations, such as selection, activation, or deactivation of various functions associated with surgical planning, intra-operative guidance, or the like. As another example, one or more cameras or other optical sensors 216 of sensors 314 may detect and interpret gestures to perform operations as described above. As a further example, sensors 314 may sense gaze direction and perform various operations as described elsewhere in this disclosure. In some examples, input devices 308 may receive manual input from a user, e.g., via a handheld controller including one or more buttons, a keypad, a touchscreen, joystick, trackball, and/or other manual input media, and perform, in response to the manual user input, various operations as described above.

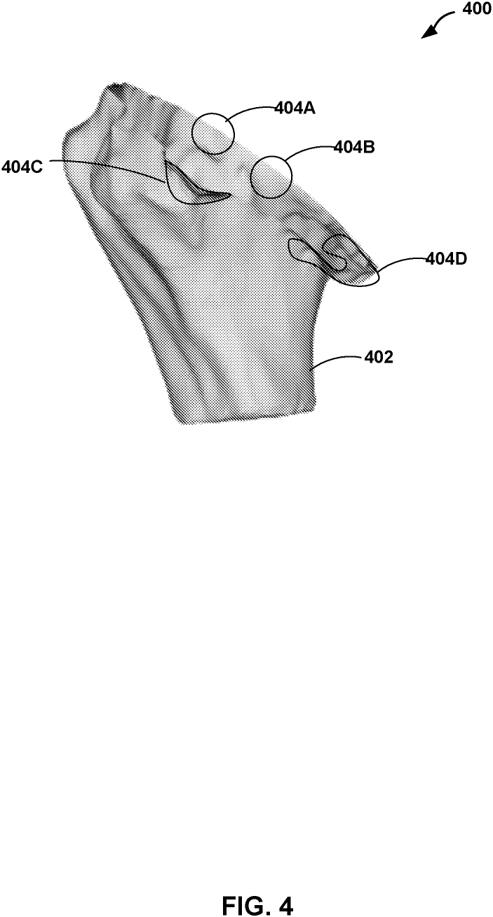

FIG. 4 is a conceptual diagram illustrating an example image data 400 generated from a medical imaging scan of patient anatomy. In some examples, image data 400 may correspond to pre-surgery image data 105 of FIG. 1. In the example, FIG. 4 illustrates humeral head 402. In some examples, image data 400 of humeral head 402 may have been obtained (e.g., captured) before a surgical procedure.

A surgeon or other medical personnel, or possibly processing circuitry 102, may use the image data 400 to identify areas 404A, 404B, 404C, and 404D (collectively, “areas 404”) prior to surgery. For example, display 110 may display image data 400 or visualization device 116 may display image data 400. The surgeon or other medical personnel may use a stylus, mouse, touchscreen or any other input device to select, trace, or otherwise mark areas 404. Areas 404 may correspond to osteophytes or other bone areas that are be removed during surgery. In some examples, processing circuitry 102 may remove the identified areas 404 from the image data to form a virtual model 106 to use as a guide during surgery. As one example, processing circuitry 102 may determine which pixels in image data 400 are within the areas 404 selected by the surgeon or other medical personnel. Processing circuitry 102 may set the color value for the pixel in the image data 400 that are within areas 404 equal to the background of image data 404 (e.g., equal to white in the example of FIG. 4). The result may be virtual model 106.

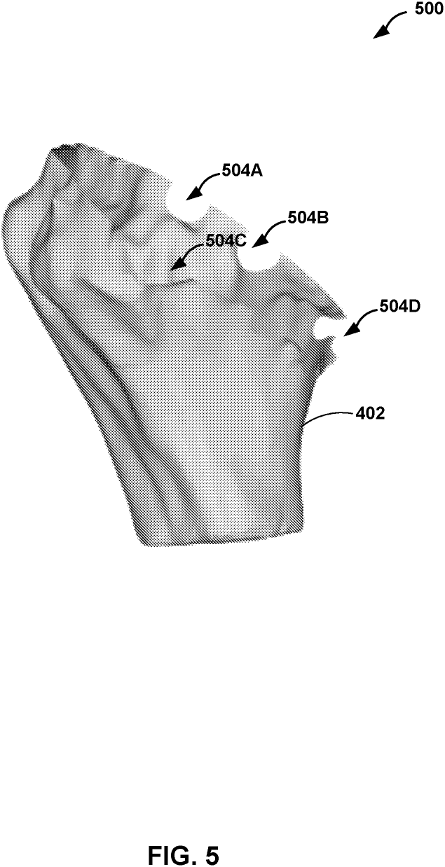

FIG. 5 is a conceptual diagram illustrating example virtual model 500 of patient anatomy with bone areas in the patient anatomy removed. In particular, FIG. 5 illustrates humeral head 402 of FIG. 4 with bone areas 504A, 504B, 504C, and 504D (collectively, “bone areas 504”) removed from the image data. That is, FIG. 5 may correspond to image data 400 of humeral head 402 with areas 404 removed. As described above with reference to FIG. 4, a surgeon or other medical personnel may select or highlight areas 404 in image data 400 and a computing device (e.g., computing device 100 of FIG. 1) may remove image data corresponding to those selected areas 404 to form virtual model 500 as shown in FIG. 5. Virtual model 500 illustrates how humeral head 402 may be altered during the initial steps of surgery and may be used by a surgeon may as a guide during the procedure. In some examples, virtual model 500 may be stored in memory 104 as virtual model 106 in FIG. 1.

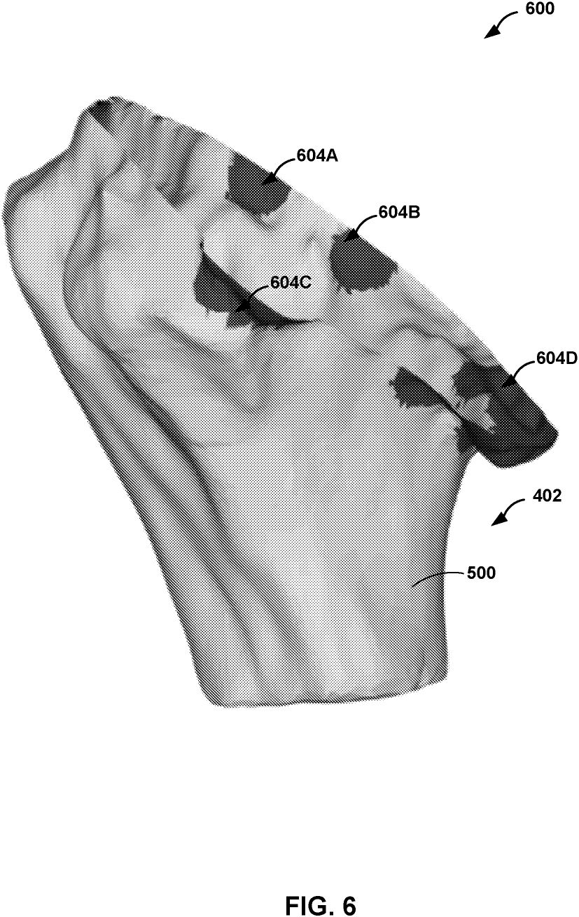

FIG. 6 is a conceptual diagram illustrating the example virtual model 500 of FIG. 5 overlaid over the patient anatomy. Although shown as being overlaid on the patient anatomy, in some examples, virtual model 500 may displayed as being proximate to the patient anatomy. For ease, the following description is described with virtual model 500 overlaid on the patient anatomy, but the example techniques are applicable to examples where virtual model 500 is displayed proximate to the patient anatomy.

In accordance with the techniques of this disclosure, processing circuitry 102 may register/align virtual model 500 to humeral head 402 in live image data 600 captured during surgery in real-time or near real-time. For example, live image data 600 is an example intra-operative image data 108 of FIG. 1. Processing circuitry 102 may register/align virtual model 500, which is an example of virtual model 106, to image data 600, which is one example of intra-operative image data 108. In this way, a surgeon may use virtual model 500 to identify areas 604A, 604B, 604C, and 604D (collectively, “bone areas 604” or “indications 604”) to remove during surgery. For instance, screen 208 of visualization device 116 may display the image data shown in FIG. 6 (e.g., bone areas 604 on virtual model 500 after virtual model 500 is aligned to image data 600).

For example, processing circuitry 102 may utilize image data 600 to register virtual model 500 to image data 600. In this way, virtual model 500 may be aligned with the actual bone that the surgeon can see in the real-world. For example, by aligning to image data 600, when visualization device 116 displays virtual model 500 and indications 604, virtual model 500 and indications 604 should be aligned with the actual bone and overlay on the actual bone. In some examples, such as where virtual model 500 and/or indications 604 are displayed as proximate to the actual bone, processing circuitry 102 may add an offset to the coordinates of virtual model 500 and/or indications 604 so that virtual model 500 and/or indications 604 are displayed proximate to the actual bone. Then, visualization device 116 may display virtual model 500 and indications 604. Indications 604 may align with the osteophytes that are on the actual bone. In this way, indications 604 identify where the osteophytes for removal are located. Then, as the surgeon removes the osteophytes, indications 604 may change color to show that surgeon’s progress in removing the osteophytes.

Processing circuitry 102 may highlight bone areas 604 (e.g., with indications 604) that are present on humeral head 402 in image data 600 that have been removed from virtual model 500. To highlight these areas, processing circuitry 102 may compare humeral head 402 in live image data 600 to virtual model 500 in real-time or near real-time to determine a difference map of virtual model 500 to humeral head 402. Processing circuitry 102 may then determine one or more characteristics (e.g., size, area, volume, shape, colors) of indications 604 to be overlaid on one or more bone areas (or volumes). Processing circuitry 102 may output virtual model 500 and indications 604 overlaid on or displayed proximate to humeral head 402 to a display (e.g., to visualization device 116 of FIG. 1) for use by a surgeon during surgery.

For example, processing circuitry 102 may highlight with a first color the parts of image data 600, which is the real-time image data, that correspond to the areas (e.g., areas 404) that were removed from pre-surgery image data 105 to form virtual model 106. The example shown in FIG. 6 illustrates the intra-operative image data 600 but before the surgeon may have actually begun to remove the osteophytes. With indications 604 (or bone areas 604), the surgeon may visualize where the osteophytes are on the anatomical object (e.g., humeral head in this example). Moreover, intra-operatively, as the surgeon begins to remove the osteophytes (e.g., as indicated by indications 604), the color of indications 604 may begin to change, and finally change to a color that indicates to the surgeon that the surgeon has removed the osteophyte to a proper level (e.g., completely removed the osteophyte).

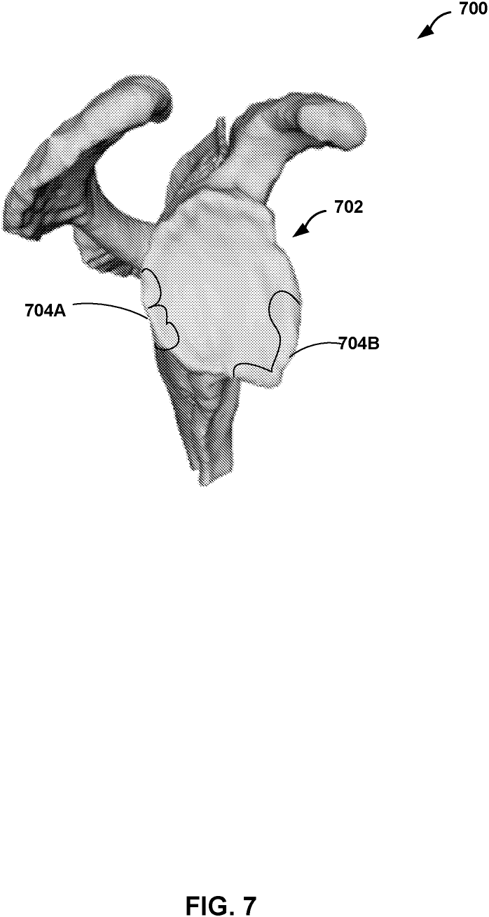

FIG. 7 is a conceptual diagram illustrating an example image data 700 generated from a medical imaging scan of patient anatomy. In some examples, image data 700 may correspond to pre-surgery image data 105 of FIG. 1. In the example, FIG. 7 illustrates scapula 702. In some examples, image data 700 of scapula 702 may have been obtained (e.g., captured) before a surgical procedure.

A surgeon or other medical personnel, or possibly processing circuitry 102, may use the image data to identify areas 704A and 704B (collectively, “areas 704”) prior to surgery. For example, the surgeon or other medical personnel may use a stylus, mouse, touchscreen or any other input device to select, trace, or otherwise mark areas 704. Areas 704 may correspond to osteophytes or other bone areas that are be removed during surgery. In some examples, processing circuitry 102 may remove the identified areas 704 from the image data to form a virtual model (e.g., like virtual model 106) to use as a guide during surgery.

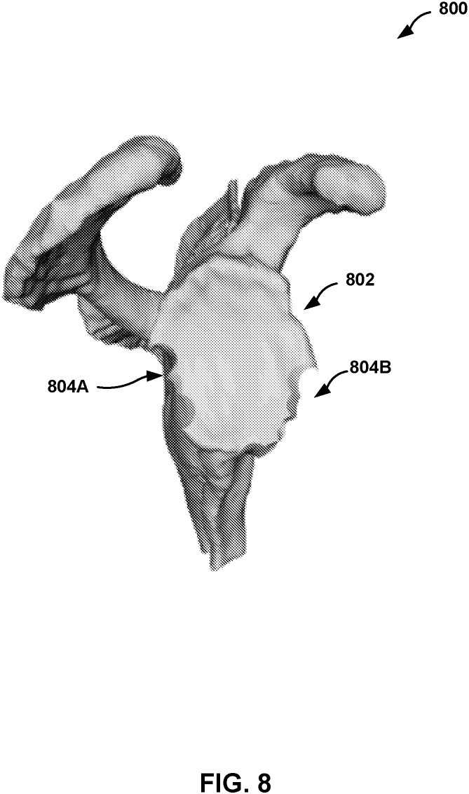

FIG. 8 is a conceptual diagram illustrating example virtual model 800 of patient anatomy with bone areas in the patient anatomy removed. In particular, FIG. 8 illustrates scapula 802, which is scapula 702 of FIG. 7 with bone areas 804A and 804B (collectively, “bone areas 804)” removed from the image data. That is, FIG. 8 may correspond to image data 700 of scapula 702 with areas 704 removed. As described above with reference to FIG. 5, a surgeon or other medical personnel may select or highlight areas 704 in image data 700 and processing circuitry 102 may remove image data corresponding to those selected areas 704 to form virtual model 800 as shown in FIG. 8. Virtual model 800 illustrates how scapula 702 may be altered during the initial steps of surgery and may be used by a surgeon may as a guide during the procedure. In some examples, virtual model 800 may be stored in memory 104 as virtual model 106 in FIG. 1.

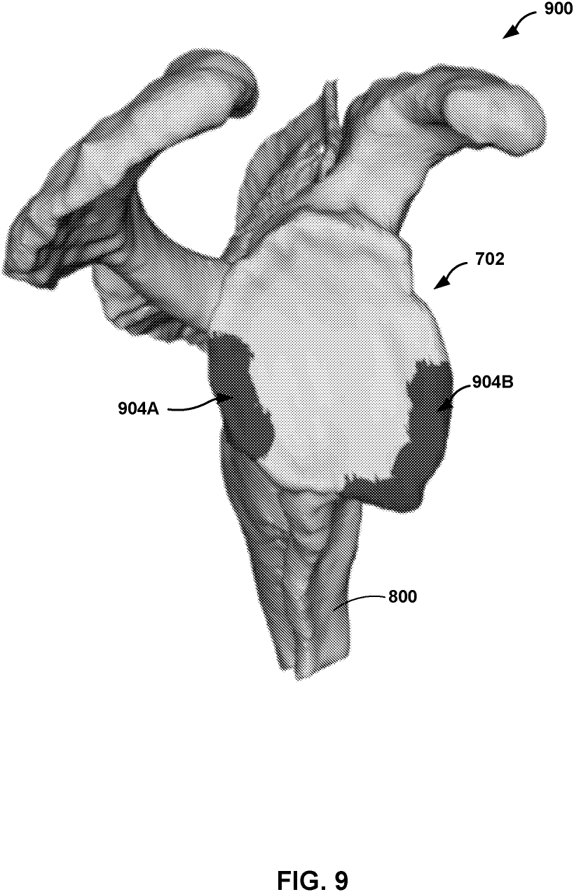

FIG. 9 is a conceptual diagram illustrating example virtual model 800 of FIG. 8 overlaid over the patient anatomy. Although shown as being overlaid on the patient anatomy, in some examples, virtual model 800 may displayed as being proximate to the patient anatomy. For ease, the following description is described with virtual model 800 overlaid on the patient anatomy, but the example techniques are applicable to examples where virtual model 800 is displayed proximate to the patient anatomy.

In accordance with the techniques of this disclosure, processing circuitry 102 may register/align virtual model 800 to scapula 702 in live image data 900 captured during surgery in real-time or near real-time. For example, live image data 90 is an example of intra-operative image data 108 of FIG. 1. In this way, a surgeon may use virtual model 800 to identify areas 904A and 904B (collectively, “bone areas 904” or “indications 904”) to remove during surgery. For instance, screen 208 of visualization device 116 may display the image data shown in FIG. 9 (e.g., bone areas 904 on virtual model 800 after virtual model 800 is aligned to image data 900).

Processing circuitry 102 may highlight bone areas 904 (e.g., with indications 904) that are present on scapula 702 in image data 900 that have been removed from virtual model 800. To highlight these areas, processing circuitry 102 may compare scapula 702 in live image data 900 to virtual model 800 in real-time or near real-time to determine a difference map of virtual model 800 to scapula 702. Processing circuitry 102 may then determine one or more characteristics (e.g., size, area, volume, shape, colors) of indications 904 to be overlaid on or displayed proximate to one or more bone areas (or volumes) of scapula 702. Processing circuitry 102 may output virtual model 800 and indications 904 to be overlaid on scapula 702 to a display (e.g., to visualization device 116 of FIG. 1) for use by a surgeon during surgery. In some examples, processing circuitry 102 may output virtual model 800 and indications 904 proximate to scapula 702 to the display for use by a surgeon during surgery.

For example, processing circuitry 102 may highlight with a first color the parts of image data 900, which is the real-time image data, that correspond to the areas (e.g., areas 704) that were removed from pre-surgery image data 105 to form virtual model 106. The example shown in FIG. 9 illustrates the intra-operative image data 900 but before the surgeon may have actually begun to remove the osteophytes. With indications 904 (or bone areas 904), the surgeon may visualize where the osteophytes are on the anatomical object (e.g., scapula in this example). Moreover, intra-operatively, as the surgeon begins to remove the osteophytes (e.g., as indicated by indications 904), the color of indications 904 may begin to change, and finally change to a color that indicates to the surgeon that the surgeon has removed the osteophyte to a proper level (e.g., completely removed the osteophyte).

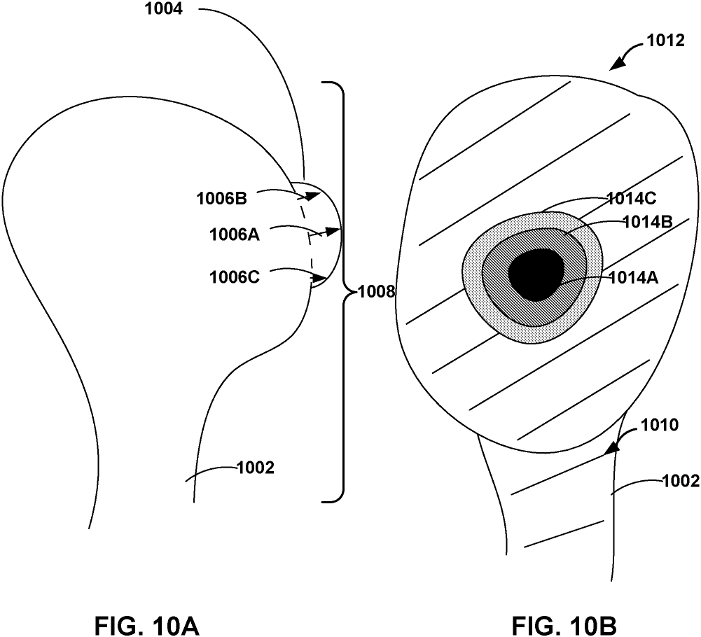

FIG. 10A is a conceptual diagram illustrating an example anatomical object 1002 (e.g., a humeral head). In this example, anatomical object 1002 includes bone area 1004 that may be removed during a medical procedure. For example, bone area 1004 may be an osteophyte that to be removed before a prosthesis may be placed during surgery.

As described above, processing circuitry 102 may obtain image data of anatomical object 1002 before the surgery (e.g., in form of pre-surgery image data 105). A surgeon or other medical personnel or processing circuitry 102 may identify bone area 1004, and processing circuitry 102 may remove bone area 1004 from the image data to form a virtual model (e.g., virtual model 106). The surgeon or other medical personnel may use a stylus, mouse, touchscreen or any other input device to select, trace, or otherwise mark bone area 1004. During surgery, processing circuitry may superimpose the virtual model over the anatomical object 1002, as described below.

FIG. 10B is a conceptual diagram illustrating example virtual model 1010 overlaid over anatomical object 1002. Although shown as being overlaid on the patient anatomy, in some examples, virtual model 1010 may displayed as being proximate to the patient anatomy. For ease, the following description is described with virtual model 1010 overlaid on the patient anatomy, but the example techniques are applicable to examples where virtual model 1010 is displayed proximate to the patient anatomy.

The example shown in FIG. 10B corresponds to the perspective of view 1008 of FIG. 10A (e.g., facing bone area 1004 of FIG. 10A). FIG. 10B is an example of image data 1012 a surgeon may view on visualization device 116 when using virtual model 1010 as a guide for removing bone area 1004 during surgery. In some examples, virtual model 1010 may be any color (e.g., green, white, clear, clear with a border).

Accordingly, the image data of anatomical object 1002 before surgery may be considered as a first image data of an anatomical object before a surgery. Processing circuitry 102 may remove areas of the anatomical object (e.g., bone area 1004) from the image data of anatomical object 1002 before surgery (e.g., first image data) to form a virtual model 1010 of anatomical object 1002 before the surgery.

Processing circuitry 102 may register, such as by aligning, virtual model 1010 to anatomical object 1002. One way to register is for processing circuitry 102 to obtain a second image data of anatomical object 1002 during surgery. Processing circuitry 102 may utilize the second image data to register virtual model 1010 so that when virtual model 1010 is displayed, virtual model 1010 is overlaid on or displayed proximate to anatomical object 1002. In other words, processing circuitry 102 may generate information of virtual model 1010 for overlay on or for displaying proximate to anatomical object 1002 based on the second image data (e.g., image data during the surgery).

As described above, virtual model 1010 may be the shape or volume of anatomical object 1002 with bone area 1004 of FIG. 10A removed. Processing circuitry 102 may compare anatomical object 1002 in image data 1012 (e.g., the second image data which is image data during the surgery) to virtual model 1010 in real-time or near real-time to determine a difference map of virtual model 1010 to the corresponding anatomical object. In one example, processing circuitry 102 may determine distances (e.g., Euclidian distances) from origin points of a normal vectors projected from virtual model to a terminal point on anatomical object in the live image data during the surgical procedure. For example, processing circuitry 102 may determine the distances of normal vectors 1006A, 1006B, and 1008C (collectively, “vectors 1006”) as shown in FIG. 10A. Vectors 1006 may represent the distances from virtual model 1010 to the top surface area of bone area 1004 (e.g., where the top surface area of bone area 1004 is determined from the segmentation). While FIG. 10A illustrates three vectors 1006, processing circuitry 102 may determine the distance from each point of virtual model 1010 to the corresponding top surfaces of bone area 1004 to determine a difference map for the areas or volumes to be removed. That is, processing circuitry 102 may determine the distance(s) of fewer or more vectors than shown in FIG. 10A.

Processing circuitry 102 may determine one or more characteristics (e.g., size, area, volume, shape, colors) of indications 1014A, 1014B, and 1014C (collectively, “indications 1014”) overlaid on or displayed proximate to bone area 1004 based on the difference map. For example, the color of each of indications 1014 may depend of the difference map. In the example shown in FIG. 10B, indication 1014A is a first color (e.g., red), indication 1014B is a second color (e.g., orange), and indication 1014C is a third color (e.g., yellow). The color of each of indications 1014 may correspond to the normal vector distance between the virtual model 1010 and anatomical object 1002 in those areas. In this example, indication 1014A in FIG. 10B may correspond to normal vector 1006A in FIG. 10A, indication 1014B in FIG. 10B may correspond to normal vector 1006B in FIG. 10A, and indication 1014C in FIG. 10B may correspond to normal vector 1006C in FIG. 10A.

In this way, processing circuitry 102 may generate one or more indications 1014 overlaid on or displayed proximate to one or more bone areas 1004 of the anatomical object 1002 corresponding to the one or more areas removed from the first image data (e.g., areas removed from the image taken before surgery that were used to generate the virtual model 1010). The shape and color of indications 1014 may guide the surgeon of the amount of bone area or volume that needs to be removed. For example, indication 1014A may indicate that more bone area 1004 must be removed under indication 1014A than under indications 1014B or 1012C. In some examples, fewer or more colors may be used. In some examples, the colors may correspond to greyscale colors, as shown in FIG. 10B. In some examples, processing circuitry 102 may determine the color for each of indications 1014 based on distance thresholds for the vectors 1006. For example, indication 1014A may be the first color because the distance of vector 1006A is beyond a first threshold, indication 1014B may be the second color because the distance of vector 1006B is beyond a second threshold but below the first threshold, and indication 1014C may be the third color because the distance of vector 1006C is beyond a third threshold but below the second threshold.

As the surgeon removes one or more portions of the bone area 1004 during surgery, processing circuitry 102 may update the difference map and indications 1014 in real-time or near real-time to other colors (e.g., red to orange to yellow to green, or any other color combinations). In this way, indications 1014 will show the updated amount of bone material that should be removed in real time or near-real time.

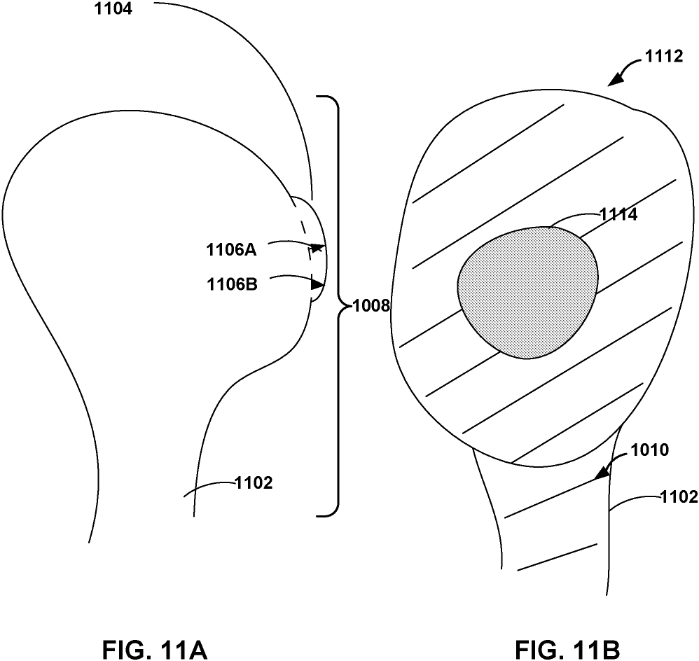

FIG. 11A is a conceptual diagram illustrating an example anatomical object 1102 (e.g., a humeral head). In this example, anatomical object 1102 includes bone area 1104. In some examples, anatomical object 1102 may correspond to anatomical object 1002 of FIG. 10A after a portion of bone area 1004 has been removed from anatomical object 1002 during surgery. That is, FIG. 11A is an example of intra-operative image data that is generated during surgery as bone area 1004 (e.g., the osteophyte) is being removed.

FIG. 11B is a conceptual diagram illustrating example virtual model 1010 overlaid over anatomical object 1102. Although shown as being overlaid on the patient anatomy, in some examples, virtual model 1010 may displayed as being proximate to the patient anatomy. For ease, the following description is described with virtual model 1010 overlaid on the patient anatomy, but the example techniques are applicable to examples where virtual model 1010 is displayed proximate to the patient anatomy.

The example shown in FIG. 10B corresponds to the perspective of view 1008 of FIG. 11A (e.g., facing bone area 1104 of FIG. 11A). FIG. 11B is an example of image data 1112 captured during surgery. In some examples, anatomical object 1102 may correspond to anatomical object 1002 of FIGS. 10A and 10B after a portion of bone area 1004 has been removed from anatomical object 1002 during surgery and the indications (e.g., indications 1014) have been updated. In particular, FIG. 10B illustrates how indications 1014 of FIG. 10B may be updated after a portion of bone area 1004 has been removed.

As described above, processing circuitry 102 may compare anatomical object 1102 in image data 1112 to virtual model 1010 in real-time or near real-time to create or update a difference map of virtual model 1010 to the corresponding anatomical object. Processing circuitry 102 may then use the updated difference map to update or generate new one or more indications (e.g., to determine one or more characteristics of the one or more indications). In the examples shown in FIGS. 11A and 11B, the computing device may determine the distances of normal vectors 1106A and 1106B (collectively, “vectors 1106”) as shown in FIG. 11A. Vectors 1106 may represent the distances from virtual model 1010 to the top of surface area of bone area 1104. While FIG. 11A illustrates two vectors 1106, processing circuitry 102 may determine the distance from each point of virtual model 1010 to the corresponding top surface of bone area 1104 to determine a difference map for areas or volumes to be removed. That is, processing circuitry 102 may determine the distance(s) of fewer or more vectors than shown in FIG. 11A.

Processing circuitry 102 may determine one or more characteristics (e.g., size, area, volume, shape, colors) of indication 1114 overlaid on bone area 1104 based on the updated difference map. In the example shown in FIG. 11B, indication 1114 corresponds to indication 1014C of FIG. 10B and corresponding indications to indications 1014A and 1014B are not shown in FIG. 11B because the length of normal vectors 1106A and 1106B (collectively, “vectors 1106”) of FIG. 11A do not exceed the first or second threshold as described above with reference to FIGS. 10A and 10B. The shape/size and color of indication 1114 may guide the surgeon of the amount of bone area or volume that still needs to be removed.

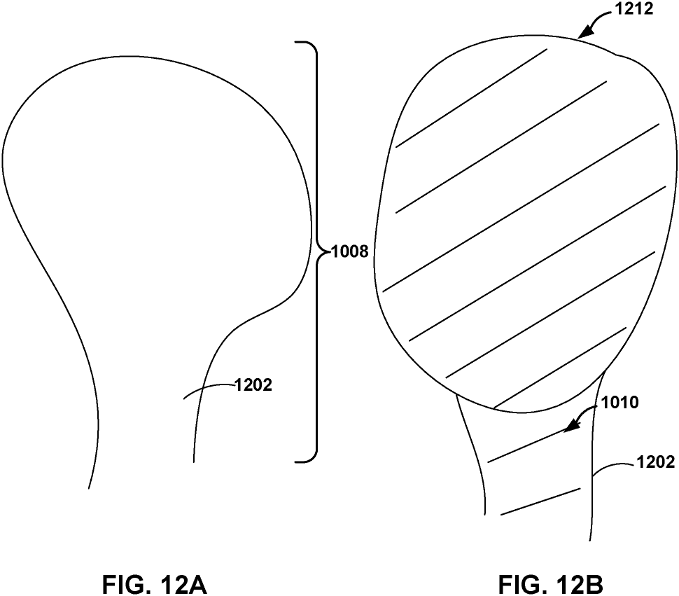

FIG. 12A is a conceptual diagram illustrating an example anatomical object 1202 (e.g., a humeral head). In this example, anatomical object 1202 may correspond to anatomical objects 1002 of FIG. 10A and 1102 of FIG. 11A after a portion of bone areas 1004 and 1104 have been removed, respectively, during surgery.

FIG. 12B is a conceptual diagram illustrating example virtual model 1010 overlaid over anatomical object 1202. Although shown as being overlaid on the patient anatomy, in some examples, virtual model 1010 may displayed as being proximate to the patient anatomy. For ease, the following description is described with virtual model 1010 overlaid on the patient anatomy, but the example techniques are applicable to examples where virtual model 1010 is displayed proximate to the patient anatomy.

The example shown in FIG. 12B corresponds to the perspective of view 1008 of FIG. 12A. FIG. 12B is an example of image data 1212 captured during surgery. In some examples, anatomical object 1202 may correspond to anatomical object 1002 of FIGS. 10A and 10B and anatomical object 1102 of FIGS. 11A and 11B after bone areas 1004 and 1104 have been removed, respectively, during surgery and the indications have been updated. In particular, FIG. 12B illustrates that indications 1014 and 1114 from FIGS. 10B and 11B have been removed after bone areas 1004 and 1104 have been removed.

As described above, processing circuitry 102 may compare anatomical object 1202 in image data 1212 to virtual model 1010 in real-time or near real-time to create or update a difference map of virtual model 1010 to the corresponding anatomical object. Processing circuitry 102 may then use the updated difference map to update or generate new one or more indications (e.g., to determine one or more characteristics of the one or more indications). In the examples shown in FIGS. 12A and 12B, the bone areas corresponding to 1004 and 1104 of FIGS. 10A and 10B and 11A and 11B have been completely removed, and thus the corresponding indications have been removed as well. In some examples, processing circuitry 102 will remove an indication when the distance of the normal vectors in the difference map are below a minimum threshold.

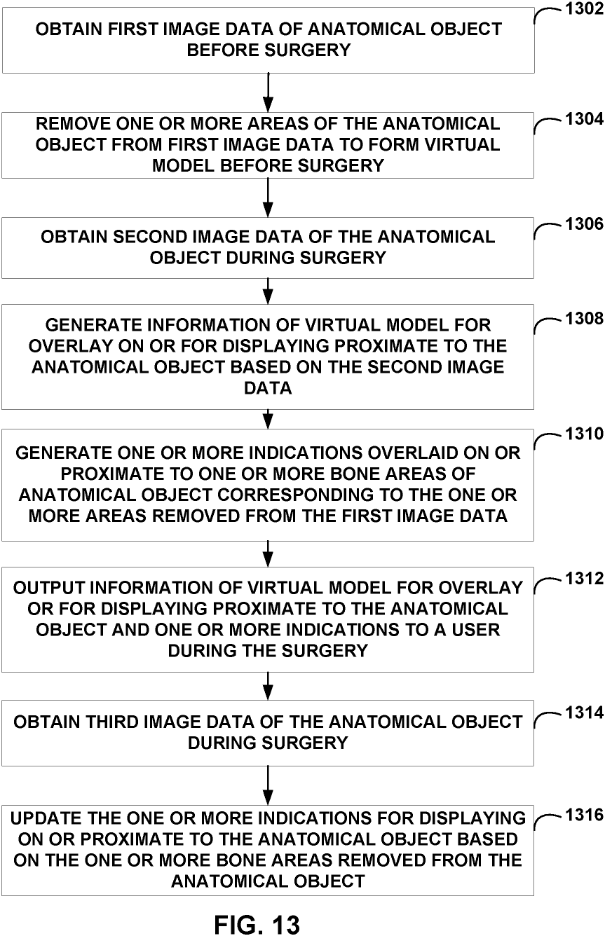

FIG. 13 is a flowchart illustrating example methods of operation in accordance with one or more example techniques described in this disclosure. For purposes of example and explanation, the method of FIG. 13 is explained with respect to processing circuitry 102 of FIG. 1. However, it should be understood that other processing circuitry may be configured to perform this or a similar method. For example, the example techniques described in this disclosure, including those of FIG. 13, may be performed by processing circuitry 102, one or more processors 202, or a combination of processing circuitry 102 and one or more processors 202.

Initially, processing circuitry 102 obtains first image data of an anatomical object before surgery (1302). For example, medical personnel (e.g., a clinician or surgeon) may take a plurality of scans (e.g., images) of the patient such as computer tomography (CT) scans) to produce first image data, such as 3D image information. One example of first image data is pre-surgery image data 105, and processing circuitry 102 may obtain the first image from memory 104.

Processing circuitry 102 may, before surgery, remove one or more areas of the anatomical object from the first image data to form a virtual model for surgery (1304). For example, processing circuitry 102 may present the first image data (e.g., pre-surgery image data 105) to medical personnel (e.g., on display 110) and the medical personnel may select or highlight the one or more areas to remove in the first image data (e.g., with a stylus, mouse, touchscreen or any other input device). Processing circuitry 102 may then remove the selected or highlighted one or more areas (or volumes) of the anatomical object in the image data to generate the virtual model (e.g., stored as virtual model 106). In this way, the surgeon or medical personnel may plan for the surgery accordingly. For example, the patient may have an injured shoulder requiring surgery, and for the surgery or possibly as part of the diagnosis, the surgeon may use the generated virtual model 106 to plan what areas to remove during surgery. In some examples, virtual model 106 may be received by device 100 from another computing device.

Processing circuitry 102 may obtain second image data of the anatomical object during the surgery (1306). One example of the second image data is intra-operative image data 108. For example, visualization device 116 may perform live image scans of the anatomical object of the patient during the surgery (e.g., using cameras, laser scanners, Doppler radar scanners, depth scanners).

Processing circuitry 102 may generate information of the virtual model for overlay on or for displaying proximate to the anatomical object based on the second image data (1308) (e.g., as shown in FIGS. 6, 9, 10B, 11B, and 12B). For example, processing circuitry 102 may register the virtual model to the anatomical object based on the second image data so that when the virtual model is displayed, the virtual model is displayed as overlay on or proximate to the anatomical object. As an example, processing circuitry 102 may determine where the virtual model is to be displayed by visualization device 116 based on aligning the virtual model to the anatomical object, and processing circuitry 102 may determine where the anatomical object is located based on the second image data.

Processing circuitry 102 may also generate one or more indications (e.g., indications 604, 904) overlaid on or proximate to the one or more areas removed from the first image data (e.g., as shown in FIGS. 6, 9, 10B, 11B, and 12B) (1310). For example, processing circuitry 102 may compare the anatomical object in second image data to the virtual model in real-time or near real-time to determine a difference map of virtual model to the corresponding anatomical object in the second image data. For example, processing circuitry 102 may determine distances (e.g., Euclidian distance) from origin points of normal vectors projected from the virtual model to terminal points on the surface of the anatomical object in the second image data and store these distances in the difference map. These distances should be non-zero values for the one or more areas that were removed from the anatomical object in the virtual model. Processing circuitry 102 may then determine, based on the difference map, one or more characteristics (e.g., size, area, volume, shape, colors) of one or more indications to be overlaid on one or more bone areas (or volumes) of the anatomical object that were removed from the virtual model.

For example, the one or more indications may be visual information that shows how much of the osteophytes remain on the bone and may provide visual information of how much of the osteophytes is to be removed. Initially, prior to surgery, the one or more indications identify the osteophytes (e.g., such as by a first color), and when the one or more indications are displayed along with the virtual model, the combined image may be similar to the first image data (e.g., pre-surgery). This may be because the virtual model is the first image data with the osteophytes removed.

Then during surgery, the surgeon may start to remove the osteophytes. In real-time or near real-time, processing circuitry 102 may receive second image data, where removal of the osteophytes has begun (e.g., such as by scrapping off the osteophytes). In this example, processing circuitry 102 may generate the one or more indications overlaid on the bone areas of the anatomical object corresponding to the areas removed from the first image data (e.g., change the color of the one or more indications to show that there is a change in the size of the osteophyte).

Processing circuitry 102 may output information of the virtual model for overlay on or for displaying proximate to the anatomical object and the one or more indications to the user during surgery (1312). For example, a surgeon may wear a visualization device (e.g., visualization device 116 of FIG. 1) during surgery and processing circuitry 102 may present the virtual model and/or the one or more indications overlaid on or proximate to the anatomical object on the visualization device in real-time or near real-time. In this way, the surgeon may be able to use the virtual model and the one or more indications as a guide for removing the one or more bone areas during surgery.

The surgeon or other medical personnel may then remove a portion of bone from the one or more areas of the anatomical object during surgery (e.g., using a scalpel, clamps, or any other medical tool or technique). Processing circuitry 102 may obtain third image data of the anatomical object (1314). That is, processing circuitry 102 will continue to obtain image data of the anatomical object in real-time or near real-time during the procedure. Processing circuitry 102 may update the one or more indications based on the one or more bone areas removed from the anatomical object (1316).

That is, processing circuitry 102 update the one or more indications as illustrated in FIG. 11B. For example, FIG. 10B may be considered as the second image, and FIG. 11B may the third image, where indications 1114 represents updated indications 1014 from FIG. 10B as the surgeon as removed more of the osteophyte (e.g., original size of osteophyte was as illustrated by 1004 and reduced size of osteophyte is illustrated by 1104).

Processing circuitry 102 may update the difference map (e.g., determine differences between points in the virtual model to points in the third image data), and update the one or more indication based on the updated difference map (e.g., update the size, area, volume, shape, and/or colors of one or more indications). This may be an iterative process where processing circuitry 102 continues to update the difference map as the surgeon removed bone areas and update the one or more indications until the difference between the virtual model and the anatomical object is below a minimum threshold value and processing circuitry 102 may remove the one or more indications (e.g., as shown in FIG. 10B). For example, the indications shown to the surgeon for removing the osteophytes may progress from the example shown in FIG. 10B to the example shown in FIG. 11B, and then to the example shown in FIG. 12B.

While the techniques been disclosed with respect to a limited number of examples, those skilled in the art, having the benefit of this disclosure, will appreciate numerous modifications and variations there from. For instance, it is contemplated that any reasonable combination of the described examples may be performed. It is intended that the appended claims cover such modifications and variations as fall within the true spirit and scope of the invention.

It is to be recognized that depending on the example, certain acts or events of any of the techniques described herein can be performed in a different sequence, may be added, merged, or left out altogether (e.g., not all described acts or events are necessary for the practice of the techniques). Moreover, in certain examples, acts or events may be performed concurrently, e.g., through multi-threaded processing, interrupt processing, or multiple processors, rather than sequentially.

In one or more examples, the functions described may be implemented in hardware, software, firmware, or any combination thereof. If implemented in software, the functions may be stored on or transmitted over as one or more instructions or code on a computer-readable medium and executed by a hardware-based processing unit. Computer-readable media may include computer-readable storage media, which corresponds to a tangible medium such as data storage media, or communication media including any medium that facilitates transfer of a computer program from one place to another, e.g., according to a communication protocol. In this manner, computer-readable media generally may correspond to (1) tangible computer-readable storage media which is non-transitory or (2) a communication medium such as a signal or carrier wave. Data storage media may be any available media that can be accessed by one or more computers or one or more processors to retrieve instructions, code and/or data structures for implementation of the techniques described in this disclosure. A computer program product may include a computer-readable medium.

By way of example, and not limitation, such computer-readable storage media can comprise RAM, ROM, EEPROM, CD-ROM or other optical disk storage, magnetic disk storage, or other magnetic storage devices, flash memory, or any other medium that can be used to store desired program code in the form of instructions or data structures and that can be accessed by a computer. Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transitory media, but are instead directed to non-transitory, tangible storage media. Disk and disc, as used herein, includes compact disc (CD), laser disc, optical disc, digital versatile disc (DVD), floppy disk and Blu-ray disc, where disks usually reproduce data magnetically, while discs reproduce data optically with lasers. Combinations of the above should also be included within the scope of computer-readable media.

Operations described in this disclosure may be performed by one or more processors, which may be implemented as fixed-function processing circuits, programmable circuits, or combinations thereof, such as one or more digital signal processors (DSPs), general purpose microprocessors, application specific integrated circuits (ASICs), field programmable gate arrays (FPGAs), or other equivalent integrated or discrete logic circuitry. Fixed-function circuits refer to circuits that provide particular functionality and are preset on the operations that can be performed. Programmable circuits refer to circuits that can programmed to perform various tasks and provide flexible functionality in the operations that can be performed. For instance, programmable circuits may execute instructions specified by software or firmware that cause the programmable circuits to operate in the manner defined by instructions of the software or firmware. Fixed-function circuits may execute software instructions (e.g., to receive parameters or output parameters), but the types of operations that the fixed-function circuits perform are generally immutable. Accordingly, the terms “processor” and “processing circuity,” as used herein may refer to any of the foregoing structures or any other structure suitable for implementation of the techniques described herein.

Various examples have been described. These and other examples are within the scope of the following claims.

1. A method comprising:
obtaining first image data of an anatomical object before a surgery;
removing one or more areas of the anatomical object from the first image data to form a virtual model of the anatomical object before the surgery;
obtaining second image data of the anatomical object during the surgery;
generating information of the virtual model for overlay on or for displaying proximate to the anatomical object based on the second image data;
generating one or more indications overlaid on or proximate to one or more bone areas of the anatomical object corresponding to the one or more areas removed from the first image data; and
outputting the information of the virtual model for overlay on or for displaying proximate to the anatomical object and the one or more indications to a user during the surgery.

2. The method of claim 1, further comprising:
obtaining third image data of the anatomical object during the surgery; and
updating the one or more indications for overlaying on or for being proximate to the one or more bone areas based on the portion removed from the one or more areas of the anatomical object.

3. The method of claim 1, wherein generating the one or more indications comprises:
determining a difference map based on a difference between the virtual model and the anatomical object in the second image data;
determining one or more characteristics for the one or more indications based on the difference map, the one or more characteristics including size, shape, or color of the one or more indications; and
outputting information of the one or more indications to a user during the surgery in a first color.

4. The method of claim 3, wherein updating the one or more indication comprises:
updating the difference map based on the difference between the virtual model and the anatomical object in the third image data;
updating the one or more characteristics for the one or more indications based on the updated difference map; and
outputting information of the one or more indications with the updated characteristics to the user during the surgery.

5. The method of claim 4, wherein updating the one or more characteristics comprises updating the color of the one or more indications to a second color, different than the first color, based on the updated difference map.

6. The method of claim 4, wherein updating the one or more characteristics comprises updating the size of the one or more indications based on the updated difference map.

7. The method of claim 4, wherein updating the one or more characteristics comprises updating the shape of the one or more indications based on the updated difference map.

8. The method of claim 1, wherein the anatomical object comprises a bone.

9. The method of claim 1, wherein the anatomical object comprises a bone, and the one or more areas of the anatomical object comprise one or more osteophytes, the method further comprising:
determining a shape of the bone without the one or more osteophytes; and
identifying the one or more osteophytes based on a difference between the first image data and the determined shape of the bone without the one or more osteophytes,
wherein removing one or more areas of the anatomical object from the first image data to form a virtual model of the anatomical object before the surgery comprises removing the one or more osteophytes based on the identification of the one or more osteophytes to form the virtual model of the anatomical object before the surgery.

10. A system comprising:
memory configured to store first image data of an anatomical object before a surgery; and
processing circuitry configured to:
obtain the first image data of the anatomical object before the surgery;
remove one or more areas of the anatomical object from the first image data to form a virtual model of the anatomical object before the surgery;
obtain second image data of the anatomical object during the surgery;
generate information of the virtual model for overlay on or for displaying proximate to the anatomical object in the second image data;
generate one or more indications overlaid on or proximate to one or more bone areas of the anatomical object corresponding to the one or more areas removed from the first image data; and
output the information of the virtual model for overlay on or for displaying proximate to the anatomical object and the one or more indications to a user during the surgery.

11. The system of claim 10, wherein the processing circuitry is configured to:
obtain third image data of the anatomical object during the surgery; and
update the one or more indications for overlaying on or being proximate to the one or more bone areas based on the portion removed from the one or more areas of the anatomical object.

12. The system of claim 10, wherein to generate the one or more indications, the processing circuitry is configured to:
determine a difference map based on a difference between the virtual model and the anatomical object in the second image data;
determine one or more characteristics for the one or more indications based on the difference map, the one or more characteristics including size, shape, or color of the one or more indications; and
output information of the one or more indications to a user during the surgery in a first color.

13. The system of claim 12, wherein to update the one or more indication, the processing circuitry is configured to:
update the difference map based on the difference between the virtual model and the anatomical object in the third image data;
update the one or more characteristics for the one or more indications based on the updated difference map; and
output information of the one or more indications with the updated characteristics to the user during the surgery.

14. The system of claim 13, wherein to update the one or more characteristics, the processing circuitry is configured to update the color of the one or more indications to a second color, different than the first color, based on the updated difference map.

15. The system of claim 13, wherein to update the one or more characteristics, the processing circuitry is configured to update the size of the one or more indications based on the updated difference map.

16. The system of claim 13, wherein to update the one or more characteristics, the processing circuitry is configured to update the shape of the one or more indications based on the updated difference map.

17. The system of claim 10, wherein the anatomical object comprises a bone.

18. The system of claim 10, wherein the anatomical object comprises a bone, and the one or more areas of the anatomical object comprise one or more osteophytes, and wherein the processing circuitry is configured to:
determine a shape of the bone without the one or more osteophytes; and
identify the one or more osteophytes based on a difference between the first image data and the determined shape of the bone without the one or more osteophytes,
wherein to remove one or more areas of the anatomical object from the first image data to form a virtual model of the anatomical object before the surgery, the processing circuitry is configured to remove the one or more osteophytes based on the identification of the one or more osteophytes to form the virtual model of the anatomical object before the surgery.

19. A non-transitory computer-readable storage medium storing instructions thereon that when executed cause one or more processors to:
obtain first image data of an anatomical object before a surgery;
remove one or more areas of the anatomical object from the first image data to form a virtual model of the anatomical object before the surgery;
obtain second image data of the anatomical object during the surgery;
generate information of the virtual model for overlay on or for displaying proximate to the anatomical object based on the second image data;
generate one or more indications overlaid on or proximate to one or more bone areas of the anatomical object corresponding to the one or more areas removed from the first image data; and
output the information of the virtual model for overlay on or for displaying proximate to the anatomical object and the one or more indications to a user during the surgery.

20. The non-transitory computer-readable storage medium of claim 19, further comprising instructions that cause the one or more processors to:
obtain third image data of the anatomical object during the surgery; and
update the one or more indications for overlaying on or for being proximate to the one or more bone areas based on the portion removed from the one or more areas of the anatomical object.

21. The non-transitory computer-readable storage medium of claim 19, wherein the instructions that cause the one or more processors to generate the one or more indications comprise instructions that cause the one or more processors to:
determine a difference map based on a difference between the virtual model and the anatomical object in the second image data;
determine one or more characteristics for the one or more indications based on the difference map, the one or more characteristics including size, shape, or color of the one or more indications; and
output information of the one or more indications to a user during the surgery in a first color.

22. The non-transitory computer-readable storage medium of claim 21, wherein the instructions that cause the one or more processors to update the one or more indication comprise instructions that cause the one or more processors to:
update the difference map based on the difference between the virtual model and the anatomical object in the third image data;
update the one or more characteristics for the one or more indications based on the updated difference map; and
output information of the one or more indications with the updated characteristics to the user during the surgery.

23. The non-transitory computer-readable storage medium of claim 22, wherein the instructions that cause the one or more processors to update the one or more characteristics comprise instructions that cause the one or more processors to update the color of the one or more indications to a second color, different than the first color, based on the updated difference map.

24. The non-transitory computer-readable storage medium of claim 22, wherein the instructions that cause the one or more processors to update the one or more characteristics comprise instructions that cause the one or more processors to update the size of the one or more indications based on the updated difference map.

25. The computer-readable storage medium of claim 22, wherein the instructions that cause the one or more processors to update the one or more characteristics comprise instructions that cause the one or more processors to update the shape of the one or more indications based on the updated difference map.

26. The non-transitory computer-readable storage medium of claim 19, wherein the anatomical object comprises a bone.

27. The non-transitory computer-readable storage medium of claim 19, wherein the anatomical object comprises a bone, and the one or more areas of the anatomical object comprise one or more osteophytes, and wherein the instructions further comprise instructions that cause the one or more processors to:
determine a shape of the bone without the one or more osteophytes; and
identify the one or more osteophytes based on a difference between the first image data and the determined shape of the bone without the one or more osteophytes,
wherein the instructions that cause one or more processors to remove one or more areas of the anatomical object from the first image data to form a virtual model of the anatomical object before the surgery comprise instructions that cause one or more processors to remove the one or more osteophytes based on the identification of the one or more osteophytes to form the virtual model of the anatomical object before the surgery.

28. (canceled)
